(19) United States
(12) Patent Application Publication
(10) Pub. No.: US 2025/0166834 A1
(43) Pub. Date: May 22, 2025

(54) System and Method for Utilizing Immersive Virtual Reality and Sensor Data in Neuromuscular Movement Coaching and Training Activities, and Physical Therapy

(71) Applicant: IMMERGO LABS, SANTA CRUZ, CA (US)

(72) Inventors: AVIV ELOR, WALNUT CREEK, CA (US); MICHAEL ORA POWELL, CAMPBELL, CA (US); ASH SEYMOUR ROBBINS, SANTA CRUZ, CA (US)

(73) Assignee: IMMERGO LABS, SANTA CRUS, CA (US)

(21) Appl. No.: 18/892,397

(22) Filed: Sep. 22, 2024

Related U.S. Application Data
(63) Continuation-in-part of application No. 17/868,743, filed on Jul. 19, 2022, now Pat. No. 12,260,010.
(63) Continuation-in-part of application No. 63/539,680, filed on Sep. 21, 2023.

(51) Int. Cl. G16H 50/30; G06N 20/00; G06T 19/00; G06V 40/20

(52) U.S. Cl. CPC G16H 50/30; G06N 20/00; G06T 19/006; G06V 40/20

(57) ABSTRACT

A system and method for utilizing virtual reality technology to provide and improve kinesiological training, analysis, and care, of a subject. Virtual reality devices may be utilized to facilitate remote coaching or physical therapy sessions in a three-dimensional virtual space whereby a digital avatar moves in accordance with the movement of a subject, i.e., a user, wearing a virtual reality device. Virtual reality devices provide and monitor exercises prescribed for the user and/or are used to record, model, and analyze kinesthetic patterns of the user to train proper form, refine technique to improve performance, and/or assess relevant physical capability such as balance or motor control, wherein the gathered data may be further analyzed to support ongoing training progress. The user may be a physical wellness client such as an athlete, a patient, a dancer, a performer, a physical therapy patient, or other suitable embodied movement subject.

### CO-PENDING PATENT APPLICATIONS

This Nonprovisional patent application is a Continuation-in-Part patent application to both (a.) Nonprovisional patent application Ser. No. 17/868,743 as filed on Jul. 19, 2022, and titled “System and Method for Utilizing Immersive Virtual Reality in Physical Therapy”; and (b.) Provisional Patent Application Ser. No. 63/539,680 as filed on Sep. 21, 2023, and titled “System and Method for Utilizing Immersive Virtual Reality and Sensor Data in Physical Therapy”. Nonprovisional patent application Ser. No. 17/868,743 and Provisional Patent Application Ser. No. 63/106,735 are hereby incorporated in entirety and for all purposes into the present disclosure.

### STATEMENT REGARDING FEDERALLY SPONSORED RESEARCH OR DEVELOPMENT

The present invention was made with U.S. Government support under Grants (Phase I & II SBIR) No. 2111847 & 2304278 awarded by the U.S. National Science Foundation for the operation of Immergo Labs, Inc. The U.S. Government has no rights to the present invention.

### BACKGROUND OF THE INVENTION

### Field of the Invention

The field of the invention relates generally to therapeutic, coaching and training applications of virtual reality technology, and specifically to use of virtual reality equipment and methods useful in physical therapy, in sports medicine, in physical training, in athletic coaching, and in movement skills training, for assessment and performance training and improvement.

### Background Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Good form in physical movement is an essential aspect of a wide variety of activities, endeavors, and disciplines, from athletics to arts, from typing to musicianship, or even merely avoiding injury and keeping oneself healthy day-to-day. Some tenets of good form may be broad and simple, such as the heavy-lifting adage of “lift with your legs, not your back”, while other nuances of good form in movement require constant practice and coaching, and can make the difference for an Olympic athlete between the silver medal and the gold. A specialist in physical motion such as but not limited to an athletic coach or a dance choreographer might spend a significant portion of a class, session, workday, or rehearsal, not merely teaching a trainee how to do a motion, but optimizing every aspect of that individual athlete or performer's posture, pacing, and form. A voice teacher or vocal coach may do the same for an orator or soloist's posture, voice placement, phrasing, breath support, and other physical aspects of vocal technique. A director or acting coach may fine-tune an actor's physicality, such as posture, character mannerisms, blocking, or other specialized physical elements of the performance such as fight choreography. A football coach may work through every beat of how a quarterback performs the motion of crouching down, catching, then throwing the ball for that all-important touchdown. Coaching and training in various skills and nuances of physical movement is integral to these kinds of activities.

Kinesiology is the scientific study of human movement, incorporating a diverse range of fields such as but not limited to biomechanics and orthopedics; strength and conditioning; sport psychology; motor control; skill acquisition and motor learning; methods of rehabilitation, such as physical and occupational therapy; ergonomics; and sport and exercise physiology. Sports and Exercise Medicine is a branch of medicine that deals with physical fitness and the treatment and prevention of injuries related to sports and exercise. Physical therapy, or physiotherapy, is the medical discipline addressing illnesses, injuries, or disabilities that limit a person's ability to move and perform functional activities in their daily life. Some reasons a patient might receive physical therapy could include recovering range of motion lost to an injury or as recovery from surgery, developing facility with a newly received prosthetic, or coping with a medical condition that affects one's proprioception or motor control such as a brain injury, stroke, Alzheimer's disease, multiple sclerosis, Parkinson's disease, or many others. A key concern in this field is assessing the patient's current motor capabilities, such as strength, flexibility, ability to balance, and motor control.

Many who perform kinesiological activities and disciplines—athletic, therapeutic, or otherwise—might agree that this kind of physical training, coaching, and care is best done in person, where explaining a physical motion is as easy as demonstrating it with one's own body; where an injured patient's body and its motion can be examined directly; where a coach and trainee can observe each other's motion from any angle or distance; where a coach might even physically adjust the trainee's body or point out a detail of the trainee's position. The medium of videos has been useful to some degree as a medium for imparting physical skills remotely; for instance, one can now watch videos online which demonstrate origami folds, yoga positions, pottery techniques, vocal techniques, safe workplace practices, and so on. However, even in a medium such as a video, or over a video call, a lot of nuance can be lost, or can only be communicated with substantially increased difficulty. Particularly in a live video call, rather than a scripted and staged training video or similar, it can be a difficult hassle for an individual live on a call such as a coach or trainee to step back from the camera, find enough space to move, make the whole movement fit inside the camera window, turn around to show multiple angles, and so on. Kinesiological endeavors such as athletic coaching and physical therapy are best done in person, and there is currently no comparably effective remote medium for such endeavors.

The field of video gaming is entertaining and interactive, and has also been shown to provide therapeutic benefits, such as honing motor skills and providing engaging challenges for players to overcome. More recent technology includes sensors and gyroscopes enabling a device to detect and codify a player's physical motion, creating such video gaming experiences as swinging a game controller like a baseball bat and having a virtual baseball on a screen ‘get hit’ and go flying as a result of the motion, or dancing with a controller and having a digitally-generated avatar perform the same dance moves. Bringing video games to practice of physical coaching and therapy is beginning to show promise in many areas, as the medium is versatile and multifaceted.

Accordingly, there is a long-felt need to apply the innovative capabilities of virtual reality consoles to the endeavors of coaching and guiding physical motion, such as but not limited to kinesiology, athletic coaching, and physical therapy.

### INCORPORATION BY REFERENCE

All publications mentioned herein are incorporated herein by reference to disclose and describe the methods and/or materials in connection with which the publications are cited. All publications, patents, and patent applications mentioned in this specification are herein incorporated by reference in their entirety and for all purposes to the same extent as if each individual publication, patent, or patent application was specifically and individually indicated to be incorporated by reference.

These incorporated publications include: (1.) U.S. Pat. No. 9,842,188 B2 titled METHOD AND SYSTEM FOR AUTOMATED MEDICAL RECORDS PROCESSING WITH CLOUD COMPUTING (Inventor David E. Stern) and issued on Dec. 12, 2017; (2.) US Patent Publication US20190214116A1, titled DIGITAL HEALTH PLATFORM FOR CHRONIC DISEASE MANAGEMENT, SECURE MESSAGING, PRESCRIPTION MANAGEMENT, AND INTEGRATED E-COMMERCE CURATION, Inventor Cheryl Lee Eberting, and published on Jul. 11, 2019; (3) US Patent Publication titled 20170329922A1 titled TELEMEDICINE PLATFORM WITH INTEGRATED E-COMMERCE AND THIRD PARTY INTERFACES, Inventor Cheryl Lee Eberting, and published on Nov. 16, 2017; (4.) US U.S. Pat. No. 10,307,060 B2, titled MESH NETWORK PERSONAL EMERGENCY RESPONSE APPLIANCE, (Inventor Tran, Bao) and issued on Jun. 4, 2019; (5.) US Patent Publication US20180114595A1, titled METHOD AND SYSTEM FOR AUTOMATED MEDICAL RECORDS PROCESSING WITH PATIENT TRACKING, (Inventor David E. Stern), and published on 2020 Jul. 14; (6.) U.S. Pat. No. 9,149,222 B1, titled Enhanced system and method for assessment of disequilibrium, balance and motion disorders, (Inventors Zets, Gary and Mortimer, Bruce) and issued on Oct. 6, 2015; (7.) U.S. Pat. No. 11,273,344 B2, titled Multimodal sensory feedback system and method for treatment and assessment of disequilibrium, balance and motion disorders, (Inventors Zets, Gary and Mortimer, Bruce) and issued on Mar. 15, 2015; U.S. Pat. No. 11,961,624 B2, titled “Augmenting clinical intelligence with federated learning, imaging analytics and outcomes decision support”, (Inventor: Smurro, James Paul) and issued on Apr. 16, 2024; and U.S. Pat. No. 10,943,025 B2, titled “Transcription data security” (Inventor: Zimmerman, Roger S.) and issued on Mar. 9, 2021.

The publications discussed or mentioned herein are provided solely for their disclosure prior to the filing date of the present application. Nothing herein is to be construed as an admission that the present invention is not entitled to antedate such publication by virtue of prior invention. Furthermore, the dates of publication provided herein may differ from the actual publication dates which may need to be independently confirmed.

### BRIEF SUMMARY OF THE INVENTION

Towards these and other objects of the method of the present invention (hereinafter, “the invented method”) that are made obvious to one of ordinary skill in the art in light of the present disclosure, what is provided is a system and method for providing coaching utilizing virtual reality technology.

In a first preferred operational practice, a user such as an athlete, trainee, performer, therapy patient, or other subject being coached, assessed, or trained in movement may access such coaching, training, assessment, etc. by using a motion capture virtual reality console such as a VR headset or similar. This coaching, training, assessment, etc. may be a pre-recorded exercise or tutorial, or may be a two-way communication session with a coach, choreographer, therapist, or other kinesiological specialist training or assessing the user and the motion of the user's body as captured by the motion capture elements of the virtual reality console and projected as a virtual reality avatar in a virtually-instantiated physical space. The technology of motion capture allows the motion of the user's body to be studied and guided in detail, and use of a second motion capture virtual reality console may allow the kinesiological specialist training or assessing the user's motion to also demonstrate motions, or interact with the user's virtual avatar (such as pointing to a part of the avatar to make a point about the user's posture or motion). This interface may allow for coaching, training, assessment, physical therapy, athletic training, dance choreography, and similar physical activities usually restricted to in-person settings to be effectively done remotely or virtually. The invented system may further provide analysis of the user's motion as captured by the motion capture, which may not be apparent otherwise, such as mechanical analysis of the user's balance and posture patterns as compared against one or more baselines.

It is understood that, in this context, the scope of the meaning of the term “user” is defined herein to include a human being, such as, but not limited to a physical wellness client, to include an athlete, a patient, a dancer, a performer, a kinesthetic performer, a trainee, a physical therapy patient, or other such subject whose motion is being captured by the invented system, so that the physical motion and performance of the user may be coached, trained, assessed, measured, and/or guided by a coach. The scope of the meaning of the term “coach” is defined herein to include, but not be limited to, a kinesiological advisor, an expert or a practitioner, or other expert in, or practitioner of, a single or multiple types of physical activity, such as but not limited to, a peer of the user, an athletic coach, a choreographer, a vocal coach, a yoga instructor, a sports medicine practitioner, a physical therapist, an occupational therapist, or any similar physical motion specialist. It is understood that the scope of meaning of the term “coach” as defined herein may comprise in part or in whole an information technology system, an expert system, or other suitable type of artificial intelligence technology system known in the art. It is further understood that these terms of “user” and “coach” are utilized throughout as defined above.

In one possible application, it is noted that a professional athlete is coached by one or more coaches, or a team of kinesiological experts such as athletic peers, athletic coaches and professionals in the area of sports medicine. The logistics of scheduling every athlete's session with every expert of their team in person can be very difficult, and if a same athlete could meet with experts remotely with comparable efficiency, the logistics of coaching could be made more efficient and/or more effective.

In a first preferred medical application of the invented method, a coach utilizes a first virtual reality device to meet virtually with a user utilizing a second virtual reality device, like a telehealth visit in virtual reality. Since so much of physical therapy pertains to observing and coaching motion of the user's body, a virtual environment wherein the coach's and user's motions are mirrored by virtual avatars may provide a more conducive medium than a two-dimensional medium such as a video telehealth call. The coach and user use the technology to observe and analyze the motion of the user's body through data generated by motion of the user's virtual reality equipment, and the coach may prescribe exercises which can be done while supervised by the coach, or “taken home” to do independently, by the user using the virtual reality equipment interface, including the possibility of providing or downloading premade physical therapy software or games, and structuring the user's “take home” exercises with these additional tools.

In an alternate preferred operational practice, a medical or physical therapy clinic or similar may set up a station including computing equipment such as a virtual reality device such as a unit comprising a headset and controllers, which may also include further elements such as a desktop computer connected to both the virtual reality device and other useful assessment equipment such as a pressure plate if present. A user, such as a physical therapy patient, may wear the virtual reality headset and hold the controllers, or use any other available assessment equipment such as a pressure plate if present, and follow directions provided by a coach or even by an interface or program presented by the VR headset screen. The user may be set tasks or challenges within the virtual environment presented by the headset, requiring movements and actions that demonstrate various levels or varieties of motor capability, all within the context of a user-friendly game played in a safe environment. In further similar implementations, the user's motion might be tracked and analyzed utilizing wearable tech, such as but not limited to IMUs, or by utilizing CVML. It is further understood that not all implementations may use or require a pressure plate, as other devices might alternatively or additionally provide insight into the user's posture and motion.

Certain preferred alternate embodiments of the invented method are designed to collate, synthesize, manage, archive and provide protected health information (hereinafter, “PHI”) to authorized health care providers, e.g., physical therapists, physicians and nursing staff, in accordance HIPAA standards, laws and regulations. Standard messaging protocol specification for exchanging structured information in the implementation of web services in computer networks.

These HIPAA-compliant inventive aspects may optionally apply Subjective Objective Assessment Plan (hereinafter “SOAP”) notes to enable synthesizing PHI and related data and for providing synthesized information that includes PHI and related data stored, transmitted, and made accessible for use by a health services provider. It is understood that PHI may include demographic information, medical histories, test and laboratory results, mental health conditions, insurance information and other data that a healthcare professional collects to identify an individual and determine appropriate care. This synthesized information may include sensor generated and data recorded from a previous session of medical therapy. The synthesized information may include audio data that is securely converted from speech to text, and may thereafter be further securely converted into SOAP format for use by the coach or therapist. In accordance with the invented method, health services provider may upload PHI that documents a user's (such as a patient's) specific diagnostic and/or treatment session notes via a webservice such as the AWS LAMBDA™ web service (hereinafter, “Lambda”™) as provided by Amazon Web Services of Seattle, WA. AWS Lambda is a serverless, event-driven compute service provided by Amazon Web Services that runs code for numerous types of software application or backend service without provisioning or managing servers. An authentication software, software service, or web service, e.g. Amazon Web Services Cognito™ (hereinafter, “Cognito”) as provided by Amazon.com, Inc. of Seattle, WA, may be used to impose HIPAA-compliant access control to PHI managed by the invented method. Additionally, HIPAA-compliant flexible document integration software, software service, or web service, again such as Cognito, may be applied in the invented method to produce, modify, and arrange documents and optionally optical character recognition files (known in the art as, “OCR files”).

It is understood that various optional aspects of the invention includes cloud storage objects to present a single unified namespace or object-space and manage desired electronic content by user or administrator-defined storage and retrieval policies. Cloud storage objects include SOAP as well as Representational State Transfer (REST), Lightweight Directory Access Protocol (LDAP) and/or Application Programming Interface (API) objects and/or other types of cloud storage objects. However, the present invention is not limited to the cloud storage objects described, and more fewer and other types of cloud storage objects can be used to practice the invention.

It is understood that the sources of biometric data preferably utilized to inform practice of embodiments of the invented method, such as to inform construction of a user health profile or notes for a coach to use, may comprise or include, but are not limited to, the following: ROM data from virtual reality equipment; ROM data from a 2D camera display; strength/torque data such as from virtual reality equipment, isokinetic machines, or dynamometers; balance data such as from virtual reality equipment, a Biodex™ virtual reality system as marketed by Biodex Medical Systems, Inc. of Shirley, NY, or a Wii balance board as marketed by Nintendo of Kyoto, Japan; heart rate or blood oxygen data such as from a FitBit™ as marketed by Google of Mountain View, CA, Apple Watch as marketed by Apple of Cupertino, CA, Garmin device as marketed by Garmin of Olathe, KS, etc, pulse oximeters, smartphones, and/or other wearable tech devices; activity and sleep data such as from wearable tech such as a FitBit™ as marketed by Google of Mountain View, CA, the Apple Health app as marketed by Apple of Cupertino, CA, a Garmin device as marketed by Garmin of Olathe, KS, a Whoop strap as marketed by WHOOP of Boston, MA, and/or other wearable tech devices; posture data such as from a user's monitor or chair, or from an Upright Go device as marketed by Upright Technologies LTD. of Tel Aviv, Israel; electroencephalography (EEG) data; electrocardiogra (EKG) data; and respiratory data such as from a spirometer.

In a preferred optional method for assessing a user's balance proficiency utilizing the invention, the user may be asked to attempt to stand perfectly still for a set length of time, such that the sensors of the virtual reality equipment (and a pressure plate, if available) can measure the extent to which the user might “sway” or “wobble” a little while standing still, and the computer can collect this data, model and analyze the detected sway pattern, and even compare this user's sway pattern against one or more baselines or profiles.

Among the anticipated benefits of the invented system and method is the aspect of providing a new physiotherapy tool that coaches can utilize as they deem necessary, as one more available option for assessment, ongoing analysis, or treatment. It is noted also that this may provide computer-enabled exercises and structure that can help users progress more easily and receive better care, while also potentially saving time for a live coach at least by providing analytic support and improving communication. Additionally, it's noted that certain users may find it easier or more comfortable to engage with and relax in a virtual environment and explore physical capabilities in that context. It's further noted that such a system may be beneficial in telehealth applications, and also that such a system, particularly with physical therapy programs designed by licensed experts preinstalled or non-local coaches available for virtual appointments, could be very useful as a vehicle for supplying improved physical therapy access to a remote location, such as for augmenting the capabilities of a general-purpose medical clinic in a rural area where actual physical therapy practitioners may be few and far between, or care otherwise limited or difficult to access.

Among the anticipated benefits of the invented system and method is the aspect of providing a tool for coaching, including remote coaching, regarding physical motion, which might also benefit other fields including but not limited to physical therapy for the injured or to increase injury prevention. Athletes who play sports and performance artists such as dancers and acrobats all are constantly trained and coached in optimal body motion at least to improve their performance and minimize chance of injury; having a reliable system for this coaching to be done remotely and well might benefit both the athletes and their coaches, and detailed computer analysis of the athlete or performer's motion, including analysis of patterns over time and comparison to other relevant motion data, may also empower these professionals—athletes, performers, and coaches—in their pursuits of athletic excellence. Providing a system for effectively performing, perceiving, analyzing, and coaching physical motion remotely, may allow for more flexible schedules and appointments between athletes and coaches, or may allow for an athlete and/or a coach who live far apart or go traveling and can't meet in person to still work together where that would otherwise have been unrealistic or inconvenient. It is noted that motion capture gear used to track and capture the motions of a healthy athlete or performer practicing a strenuous physical routine may need more or different securing or placement than gear being used to capture the motion of an injured person standing still or performing gentle motions for physical therapy.

It is further noted that there are a wide variety of activities which include physical aspects but aren't categorized as athletic, and it is noted and understood that a proficient expert in teaching these varieties of physical skills may also be considered a coach as defined herein. One example might be performance arts including dance, acting, and singing; obviously a choreographer or dance coach will have critiques for a performer's movement technique and form, but also, a vocal coach or acting coach will extensively develop a performer's breath support, vocal placement, posture, physicality, and so on. Another example might be physical skills used in cooking and making, such as but not limited to tossing pizza dough, separating egg whites, using a pottery wheel, or drawing strokes in painting and calligraphy; one generally learns these techniques from watching or being instructed by someone with more expertise in the relevant discipline. Other examples may include driving or piloting a vehicle, or skills associated with working in specific workplaces, such as operating specialized machinery, safely handling heavy lifting, or practicing good technique in typing for reduced risk of carpal tunnel injury.

Certain preferred embodiments of the invention may include a client device comprising at least: an augmented reality user set (“user set”), the user set comprising a head set and an additional positional feedback device, wherein the user set is configured to be worn by a human user and to generate and transmit positional information related to a dynamic positioning of the human user's body; one or more processors bi-directionally communicatively coupled by a communications module with the user set; a memory bi-directionally communicatively coupled by the communications module with the one or more processors and the user set, the memory storing software executable instructions executing on the client device, the software executable instructions when executed by the one or more processors cause the client device to: access a video segment directing the client system to dynamic visual rendering of a user avatar derived from and dynamically responsive to positional information received from the user set; transmit to the headset a sequence of data frames from a data stream program, wherein each data frame including positioning information of a modeling avatar for rendering by the headset, the modelling avatar adapted to dynamically present to the user via the headset aspects of a personalized therapeutic movement path; and display an interactive dynamic overlay of the modelling avatar over the user avatar by the headset, the interactive overlay displayed in association with dynamically updated positional information, wherein the dynamically updated position information of the user set is derived from the positional data generated by the user set and received by the one or more processors.

This client device might also include the data stream program containing information derived from dynamically generated positional data received previous to a therapeutic session. The client device might also include the data stream program containing information that modifies a dynamic instanton of the modeling avatar on the basis of dynamically generated positional data received from the user set during a same therapeutic session. The client device might also include the data stream program containing information that modifies a dynamic instanton of the modeling avatar on the basis of dynamically generated positional data received from the user set during an earlier observed therapeutic session.

The user set may dynamically generate the positional data received previous to the therapeutic session. The data stream program may contain information describing a full range of preferred motion personalized for an identified user. The data stream program may contain information describing a modified range of a preferred limited range of motion personalized for an identified user. The data stream program may contain information enabling the client device to dynamically vary a rendered range of motion on the bases of positional data received within a same therapeutic session. The client device may further comprise the one or more processors receiving an informational update to the data stream program, wherein the informational update provides information to the client device that enables a revision of the positioning information of a modeling avatar adapted for rendering by the headset. The update information may be received and applied by the client device during the same therapeutic session. The memory may contain additional software executable instructions that when executed by the one or more processors enable the client device to provide alternate avatar positioning information of two or more modeling avatars for rendering by the headset, wherein each modeling avatar is adapted to dynamically present to the user via the headset aspects of an alternate personalized therapeutic movement path.

At least a portion of the software executable instructions may be received and applied during the same therapeutic session. The client device may be adapted to and at least partially renders a virtual reality environment by means of the user set.

The device may further comprise: the user set further comprising an audio data rendering module (“audio module”) coupled with the one or more processors; additional instructions of the software executable instructions that when executed by the one or more processors cause the client device to transmit audio data to the audio module. The audio data may be generated from the data stream program. The audio data may be sourced externally from the client device. Each data stream program of the client device may include a respective video sequence information and a respective audio sequence information, and the audio sequence information is adapted for rendering by the user set audio module. The client device may include the interactive dynamic overlay of the modeling avatar including a Hypertext Markup Language (HTML) overlay.

An invented method of rendering interactive overlays within a therapeutic session may comprise the following: accessing by one or more processors a video segment directing a client system to dynamic visual render a user avatar at a headset information partially derived from and dynamically responsive to positional information received from the headset; transmitting to the headset a sequence of data frames from a data stream program, wherein each data frame including positioning information of a modeling avatar for rendering by the headset, the modeling avatar adapted to dynamically present to the user via the headset aspects of a personalized therapeutic movement path; displaying an interactive dynamic overlay of the modeling avatar over the user avatar by the headset, the interactive overlay displayed in association with dynamically updated positional information, wherein the dynamically updated position information of the user set is derived from the positional data generated by the user set and received by the one or more processors. This method might further comprise establishing, by the one or more processors, a communication channel with a session management system, and wherein at least a portion of the sequence of data frames rendered by the headset are received by client device over the established communication channel. The method might further include the one or more processors being adapted to at least partially render a virtual reality environment by means of the headset.

Further additional or alternative aspects of the invention might include a non-transitory computer-readable medium comprising computer code instructions stored thereon, the computer code instructions, when executed by one or more processors, cause the one or more processors to: access a video segment directing a client system to dynamic visual render a user avatar at a headset information partially derived from and dynamically responsive to positional information received from the headset; transmit to the headset a sequence of data frames from a data stream program, wherein each data frame including positioning information of a modeling avatar for rendering by the headset, the modeling avatar adapted to dynamically present to the user via the headset aspects of a personalized therapeutic movement path; display an interactive dynamic overlay of the modeling avatar over the user avatar by the headset, the interactive overlay displayed in association with dynamically updated positional information, wherein the dynamically updated position information of the user set is derived from the positional data generated by the user set and received by the one or more processors.

Certain embodiments may include or comprise a method for training and applying a machine learning algorithm to assess and predict physical health of a human user based on measurement of physical motion of the human user, the method comprising: assessing and categorizing a physical health state of a sample user; providing a first motion capture system comprising a force plate, a virtual reality headset, and two handheld virtual reality controllers; instructing the sample user to stand on the force plate, wear the virtual reality headset, and grip the two handheld virtual reality controllers; instructing the sample user to stand as still as the sample user is able to, for a plurality of short durations of time, while the motion capture system records a set of sample user motion data regarding slight bodily motion of the sample user even while the sample user is attempting to stand still; filtering and batch processing the set of sample user motion data; training a machine learning algorithm to predict a sway pattern of the sample user based on the set of sample user motion data; training a machine learning algorithm to associate the sway pattern of the set of sample user motion data as characteristic for the physical health state of the sample user as previously assessed; exporting the machine learning algorithm as trained to a virtual reality application; and applying the machine learning algorithm as trained to predict an unknown physical health state of a new user based on assessment of a new user sway pattern in a received motion data set associated with the new user.

The method may further include the assessment of the new user being done remotely via an electronic communications network. The method may further include the output of the virtual reality headset and two handheld virtual reality controllers being received when in use by the new user and analyzed by application of the machine learning algorithm. The method may include the sample user being an athlete. The method may include the new user being an athlete. The method may include the new user acting under advice of a coach. The method may further comprise: assessing and categorizing a physical health state of each of a plurality of sample users; providing a same motion capture system serially to each of the plurality of sample users, the motion capture system comprising a force plate, a virtual reality headset, and two handheld virtual reality controllers to each of the sample users; instructing each sample user to separately and individually stand on the force plate, while wearing one the motion capture system, and separately grip the two handheld virtual reality controllers; instructing each sample user to stand as still as each sample user is able to, for a plurality of short durations of time, while the motion capture system records comprising a set of sample user motion data regarding slight bodily motion of each sample user even while each sample user is attempting to stand still and monitored by the motion capture system; and filtering and batch processing the set of sample user motion data. The method may further comprise adding at least one set of sample data that is generated by use of at least one sample user of an alternate motion capture system, the alternate motion capture system comprising an alternate force plate, an alternate virtual reality headset, and two alternate handheld virtual reality controllers. The method may further comprise: assessing and categorizing a physical health state of each of a plurality of sample users; providing a plurality of motion capture systems comprising a force plate, a virtual reality headset, and two handheld virtual reality controllers to each of the sample users; instructing each sample user to separately and individually stand one of the plurality of force plates, while wearing one of the plurality of motion capture systems, and grip the two handheld virtual reality controllers of said one of the plurality of motion capture systems; instructing each sample user to stand as still as each sample user is able to, for a plurality of short durations of time, while one of said plurality of motion capture system records one of a set of sample user motion data regarding slight bodily motion of each sample user even while each sample user is attempting to stand still; and filtering and batch processing the set of sample user motion data. The method may further comprise the virtual reality headset rendering a same virtual reality session to at least two sample users while sample data is recorded. The method may further comprise the virtual reality session being derived from the virtual reality application. The method may further comprise the virtual reality headset rendering the same virtual reality session to the new user while data is recorded. The method may further comprise the virtual reality session being derived from the virtual reality application.

Certain embodiments might comprise or include a client device comprising: an augmented reality user set (“the user set”), the user set comprising a headset and an additional positional feedback device, the positional feedback device comprising a plurality of body element positional sensors (“the plurality of sensors”) communicatively coupled with the headset, wherein the user set is configured to be worn by a human user and to generate and transmit relative body part dynamic positional information describing a dynamic kinesiologic action of the user's body; one or more processors bi-directionally communicatively coupled by a communications module with the user set; and a memory bi-directionally communicatively coupled by the communications module with the one or more processors and the user set, the memory storing software executable instructions executing on the client device, the software executable instructions when executed by the one or more processors cause the client device to: access a video segment directing the client system to dynamically visually render a user avatar derived from and dynamically responsive to kinesiologic relative body element positional information generated by and received from the plurality of sensors; transmit to the headset a sequence of data frames from a data stream program, the data stream program, the data stream program presenting at least one personalized body parts movement pathway (“the pathway”) indicating at least one recommended kinesiologic path of at least two internal anatomical elements of the user, wherein the sequence of data frames provides kinesiologic and positioning information of a modeling avatar for rendering by the headset, the modeling avatar adapted to dynamically present to the user via the headset aspects of the at least one personalized movement pathway; and display an interactive dynamic overlay of the modeling avatar over the user avatar by the headset, the interactive overlay displayed in association with a plurality of dynamically updated kinesiologic body part positional information received by the headset from the plurality of sensors, wherein the dynamically updated kinesiologic body part positional information generated by the user set is derived from the plurality of dynamically kinesiologic body part positional information generated by the plurality of sensors of the positional feedback device and received and integrated into the user avatar by the one or more processors. The method may further comprise the video segment comprising a sequence of athletic movement images. The method may further comprise the video segment comprising a sequence of human performance movement images. The method may further comprise the sequence of human performance movement images expressing a choreographed pattern of human movement. The method may further comprise the client device being accessed by a human user for a health evaluation. The method may further comprise the client device being accessed by a human user for a movement training session. The method may further comprise the client device being accessed by a human user for a performance training session.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE SEVERAL VIEWS OF THE DRAWING(S)

The detailed description of some embodiments of the invention is made below with reference to the accompanying figures, wherein like numerals represent corresponding parts of the figures.

FIG. 1A is a diagram presenting a first embodiment of an invented system for utilizing VR technology in physical coaching, comprising at least a virtual reality device, and optionally further including a computing device and/or an optional force plate;

FIG. 1B is presenting a minimal version of the system of FIG. 1A;

FIG. 2 is a diagram presenting an electronic communications network connecting elements of a second embodiment of an invented system for utilizing VR technology in physical coaching;

FIG. 3A is a hardware diagram presenting the computing device of FIG. 1A;

FIG. 3B is a hardware diagram presenting the virtual reality device of FIG. 1A and FIG. 1B;

FIG. 3C is a hardware diagram presenting the second virtual reality device of FIG. 2;

FIG. 4 is a first chart presenting various aspects of the invented method as practiced within the invented system of FIG. 1A, particularly pertaining to forming connections between user actions and digital modeling of the virtual reality gear;

FIG. 5 is a second chart presenting various aspects of the invented method as practiced within the invented system of FIG. 2, particularly pertaining to user and coach interaction within a virtual 3D interface;

FIG. 6 is a third chart presenting various aspects of the invented method as practiced within the invented system of FIG. 1A;

FIG. 7 is a fourth chart presenting various aspects of the invented method as practiced within the invented system of FIG. 1A, pertaining particularly to analysis and assessment of a user's joint health;

FIG. 8 is a fifth chart presenting various aspects of the invented method as practiced within the invented system of FIG. 1A, pertaining particularly to capturing user data over time;

FIG. 9 is a sixth chart presenting various aspects of the invented method as practiced within the invented system of FIG. 1A, particularly pertaining to flow exercise guidance;

FIG. 10 is a seventh chart presenting various aspects of the invented method as practiced within the invented system of FIG. 1A, particularly pertaining to a coach and user session;

FIG. 11 is an eighth chart presenting various aspects of the invented method as practiced within the invented system of FIG. 1A, particularly pertaining to full body analysis;

FIG. 12 is a process chart presenting one preferred procedure for assessing a user's balance capability utilizing the system of FIG. 1A;

FIG. 13 is a first process chart presenting a first process for synthesizing recorded data into notes for use by a coach and as additionally enabled at least partially by the network of FIG. 2 and/or FIG. 18;

FIG. 14 is a second process chart presenting a second process for synthesizing recorded data into notes for use by a coach and as additionally enabled at least partially by the network of FIG. 2 and/or FIG. 18;

FIG. 15 is a third process chart presenting a third process for synthesizing recorded data into notes for use by a coach and as additionally enabled at least partially by the network of FIG. 2 and/or FIG. 18;

FIG. 16 is a fourth process chart presenting a fourth process for synthesizing recorded data into notes for use by a coach and as additionally enabled at least partially by the network of FIG. 2 and/or FIG. 18;

FIG. 17 is a fifth process chart presenting a fifth process for synthesizing recorded data into notes for use by a coach and as additionally enabled at least partially by the network of FIG. 2 and/or FIG. 18;

FIG. 18 is a block diagram of an enhanced embodiment of the information technology network of FIG. 2, and by which the one or more, or all of the aspects of the processes for synthesizing recorded data of FIG. 13 through FIG. 17 are practiced;

FIG. 19 is a block diagram representing a training data set for use in the machine learning algorithm training process of FIGS. 20 through 23;

FIG. 20 is a process chart presenting an overview of a preferred method for training and utilizing a machine learning algorithm utilizing the system of FIG. 1A;

FIG. 21 is a first process chart presenting further detail regarding the training data generation aspect of the process chart of FIG. 20;

FIG. 22 is a second process chart presenting further detail regarding the machine learning algorithm training aspect of the process chart of FIG. 20; and

FIG. 23 is a third process chart presenting further detail regarding the aspect of utilizing the trained machine learning algorithm of the process chart of FIG. 20.

### DETAILED DESCRIPTION OF THE INVENTION

In the following detailed description of the invention, numerous details, examples, and embodiments of the invention are described. However, it will be clear and apparent to one skilled in the art that the invention is not limited to the embodiments set forth and that the invention can be adapted for any of several applications.

It is to be understood that this invention is not limited to particular aspects of the present invention described, as such may, of course, vary. It is also to be understood that the terminology used herein is for the purpose of describing particular aspects only, and is not intended to be limiting, since the scope of the present invention will be limited only by the appended claims. Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the range's limits, an excluding of either or both of those included limits is also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements, or use of a “negative” limitation.

When elements are referred to as being “connected” or “coupled,” the elements can be directly connected or coupled together or one or more intervening elements may also be present. In contrast, when elements are referred to as being “directly connected” or “directly coupled,” there are no intervening elements present.

In the specification and claims, references to “a processor” include multiple processors. In some cases, a process that may be performed by “a processor” may be actually performed by multiple processors on the same device or on different devices. For the purposes of this specification and claims, any reference to “a processor” shall include multiple processors, which may be on the same device or different devices, unless expressly specified otherwise.

The subject matter may be embodied as devices, systems, methods, and/or computer program products. Accordingly, some or all of the subject matter may be embodied in hardware and/or in software (including firmware, resident software, micro-code, state machines, gate arrays, etc.) Furthermore, the subject matter may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. By way of example, and not limitation, computer readable media may comprise computer storage media and communication media.

Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an instruction execution system. Note that the computer-usable or computer-readable medium could be paper or another suitable medium upon which the program is printed, as the program can be electronically captured, via, for instance, optical scanning of the paper or other medium, then compiled, interpreted, of otherwise processed in a suitable manner, if necessary, and then stored in a computer memory.

When the subject matter is embodied in the general context of computer-executable instructions, the embodiment may comprise program modules, executed by one or more systems, computers, or other devices. Generally, program modules include routines, programs, objects, components, data structures, etc. that perform particular tasks or implement particular abstract data types. Typically, the functionality of the program modules may be combined or distributed as desired in various embodiments.

Additionally, it should be understood that any transaction or interaction described as occurring between multiple computers is not limited to multiple distinct hardware platforms, and could all be happening on the same computer. It is understood in the art that a single hardware platform may host multiple distinct and separate server functions.

Throughout this specification, like reference numbers signify the same elements throughout the description of the figures.

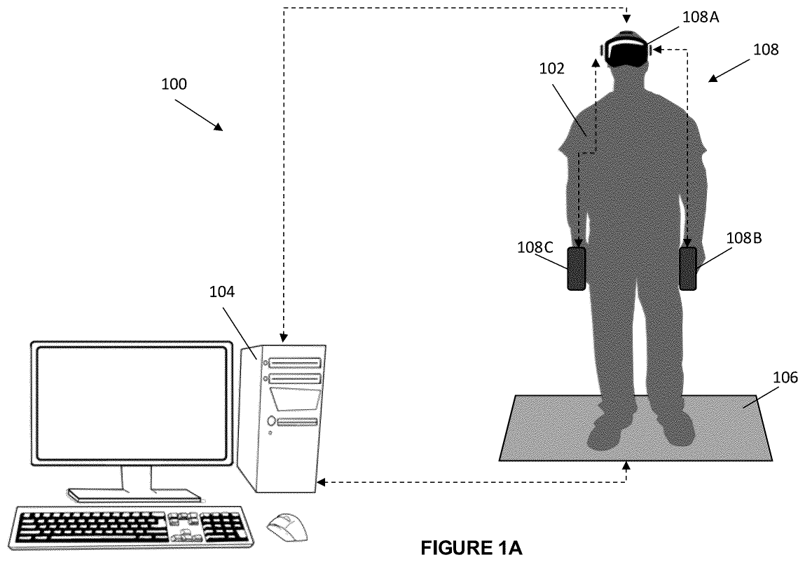

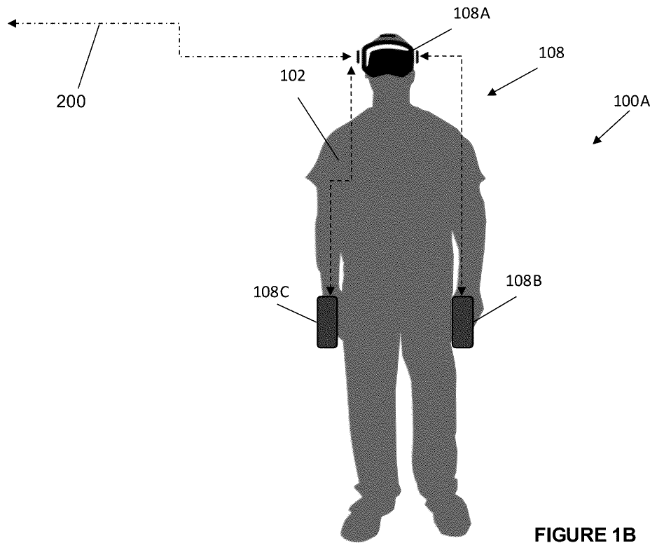

Referring now generally to the Figures, and particularly to FIG. 1A, FIG. 1A is a diagram presenting a basic overview of hardware aspects of an invented movement assessment system 100 (“the system 100”), as operated by a user 102. It is noted that the user 102, such as a therapeutic patient or a movement trainee. in this context might be any individual whose movement is being assessed, monitored, analyzed, or recorded by the system 100, such as but not limited to a physical therapy patient receiving diagnosis or treatment, an athlete being coached in movements relevant to the athlete's sport or field, a performance artist being coached in movements relevant to the performer's art or profession, or a coach or technician programming the system 100 (such as by demonstrating movements for the system 100 to record).

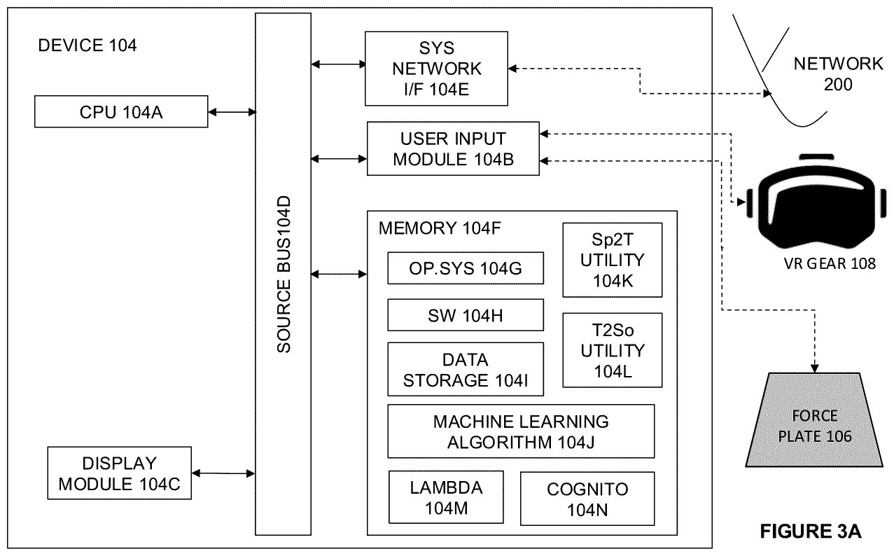

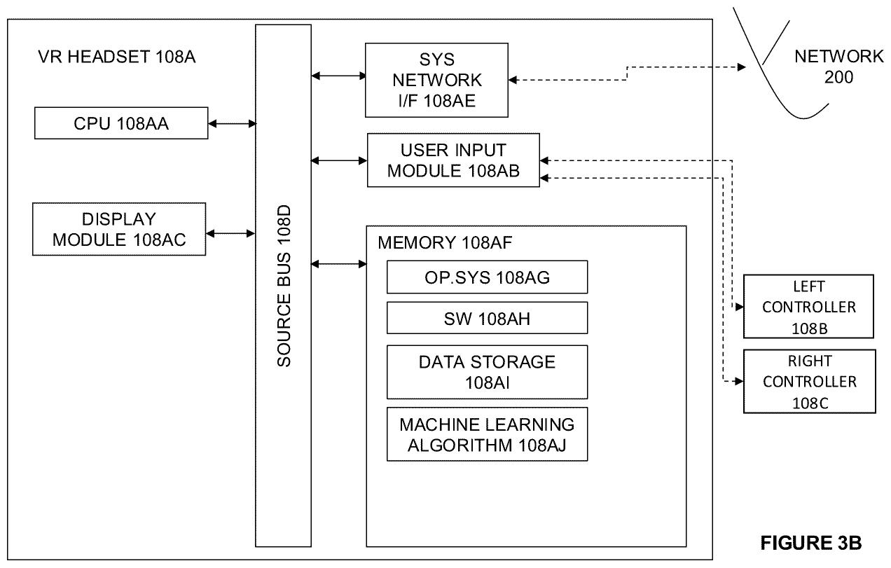

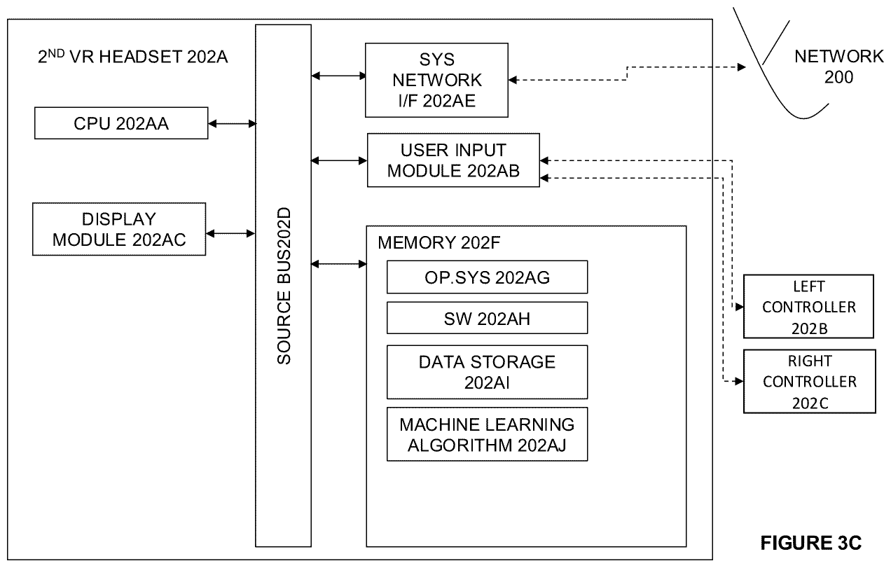

The system 100 may include at least some or all of the following hardware: a computing device 104 (“the device 104”) such as but not limited to a desktop computer or laptop, an optional force plate 106, and a set of virtual reality gear 108 (“the VR gear 108”) such as the model presented here which further comprises a VR headset 108A, a left VR controller 108B, and a right VR controller 108C (“the VR controllers 108B&C”). In preferred operation, the user 102 puts on the VR gear 108 and stands on the optional force plate 106 if included, so that the motions of the user 102 as directed by a coach (not shown) or by the VR gear 108 interface can be captured by the device 104 via the optional force plate 106 and the VR gear 108 and analyzed. It is noted that the VR gear 108 may also itself be a computing device, and may be capable of performing certain aspects or embodiments of the invented method without interfacing with a second computing device as shown here. In that case, the VR gear 108 might be understood to perform the roles of both the VR gear 108 (e.g. being worn by the user and performing motion capture) and also the device 104 (e.g. receiving, analyzing, storing, and displaying data, such as for a coach to interpret). Alternatively, as presented in FIG. 3A, the VR gear 108 may be merely a peripheral device of the device 104, analogous to a game controller and having no computing power of its own.

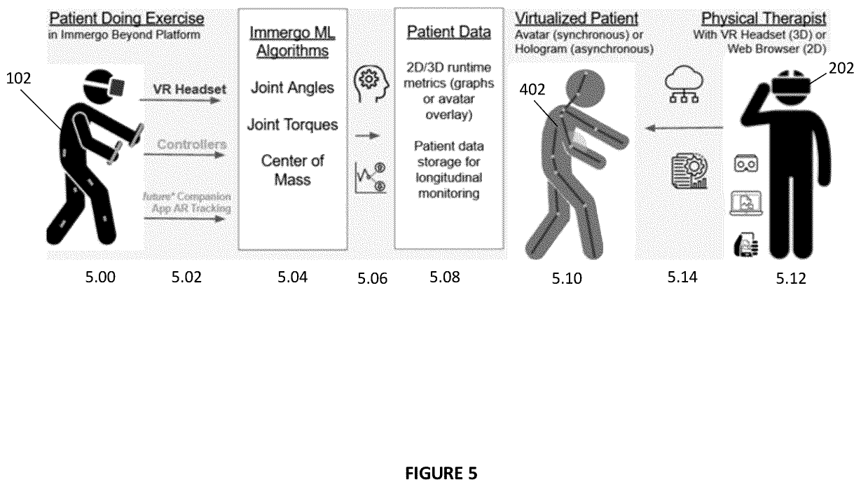

It is understood that the optional force plate 106 preferably comprises a suitable clinical grade force plate known in the art, and may be used primarily for data collection to train algorithms, such as those of FIG. 5, and it may not be necessary for the user 102 to use or have the optional force plate 106 included or available for therapeutic use at a clinic, at the user's home, or other suitable location.

It is further understood that any equipment indicated herein as used by the user 102 might be considered as optional or interchangeable, understanding that the equipment the user 102 has access to may vary, and applications may accordingly vary based on what can be assessed and done using the available equipment. This may range from a clinical setting wherein the preferred equipment may be provided for this purpose, to a home setting wherein the user 102 may not own a VR headset, force plate, etc.

It is noted that, depending upon the application and hardware capability, the VR gear 108 alone may often perform some or all of the preferred aspects of the invented method and minimize or even eliminate the necessity of including external devices such as the computing device 104 or the optional force plate 106 in every implementation; for the sake of completeness, these items are still presented herein sometimes where they might optionally or sometimes be included. It is further noted that force plates in particular are generally expensive and difficult to obtain, set up, maintain, and operate, and that making physical therapy at least as accessible as a VR headset is, lowering barriers to providing and accessing care that may result from distance or expense, is a notable benefit of preferred embodiments of the invented system and method. Accordingly, some more established implementations, such as lab settings or large physical therapy practices, might set up a more ‘comprehensive’ version of the system 100, such as one with the optional force plate 106 and additional devices or computers attached, and may generate more complete or nuanced data that way, while machine learning based on some of that high-quality data may benefit or empower AI analysis of comparatively less comprehensive data gathered by the VR gear 108 alone as utilized by a smaller practice or by an individual accessing care or coaching remotely.

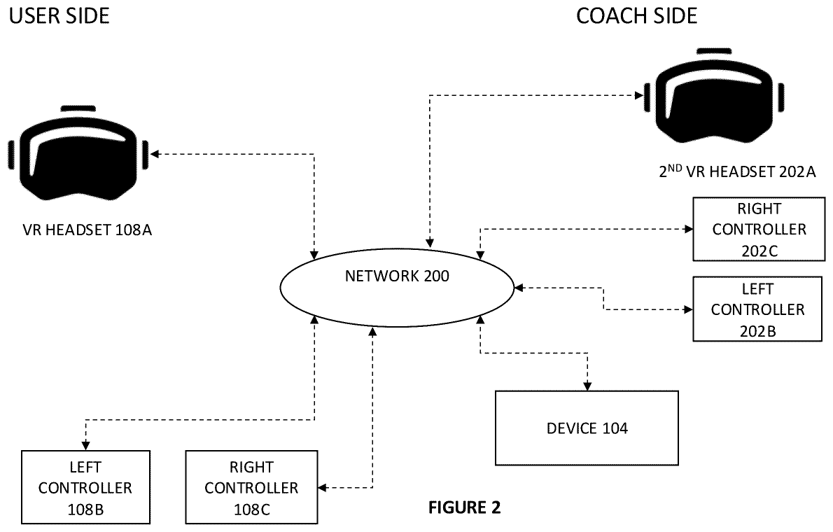

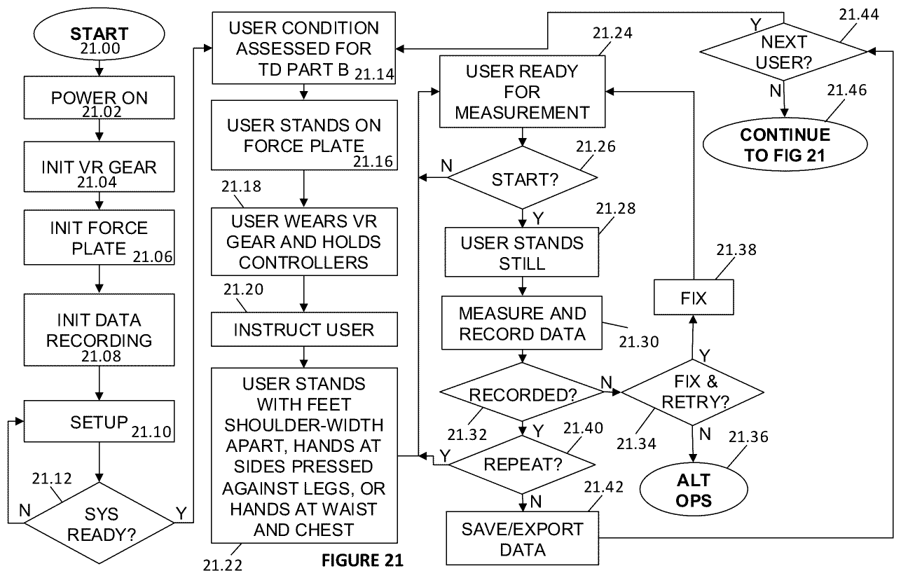

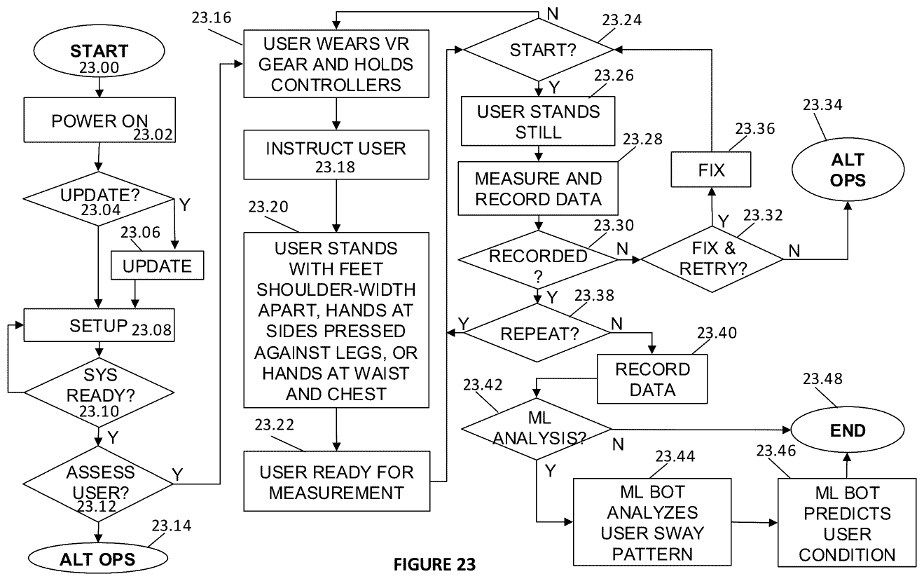

Referring now generally to the Figures, and particularly to FIG. 1B, FIG. 1B is a diagram presenting the user 102 operating a minimal embodiment of the system 100, a minimal system 100A, comprising just the VR gear 108 connected to the network 200 of FIG. 2. It is noted that the system 100 with all components may be an embodiment of the system 100 used in a laboratory setting for gathering data, such as per the method of FIG. 21, while the minimal system 100A embodiment may be suited to application by a private individual user 102, such as an athlete or PT patient using their own personal VR gear at home to perform the process of FIG. 23. It is further understood that the methods described herein might generally be performed using a full setup of the system 100, and might also be possible with a variation or subset of the full setup, such as an embodiment omitting the optional force plate 106 but nothing else, or the minimal system 100A as presented here.

Referring now generally to the Figures, and particularly to FIG. 2, FIG. 2 is a diagram presenting an electronic communications network 200 (“the network 200”) connecting a plurality of devices in a second preferred embodiment of a system for practicing aspects of the invented method. It is noted that, while the system 100 might be viewed as an environment for practicing the invented method which may not necessarily require much networking besides direct connections between devices, such as a station set up in a coach's office for the user 102 to use during an in-person appointment, the network 200 of FIG. 2 would be a preferred environment for practicing the invented method either in person or remotely. The devices connected to the network 200 of FIG. 2, which might be any kind of electronic communications network such as but not limited to the internet or a local area network (LAN), are broadly categorized into a ‘user 102 side’ which may include the VR headset 108A, the left VR controller 108B, the right VR controller 108C, and a ‘coach side’ which may include the device 104 and also a second VR headset 202 further comprising a second VR headset 202A, a second left VR controller 202B, and a second right VR controller 202C (also, “the 2nd VR controllers 202B&C”), which may be worn and utilized by a coach interacting with the user 102. It is noted that, depending on particular device and network configurations, the VR controllers may connect directly to the VR headsets or have their own ‘channel’ for connecting with other elements of a same set of VR gear, or a set of VR gear may rely on a network connection such as WiFi or Bluetooth to make these connections; these Figures present each of these possibilities in the various included Figures to reflect this variable connectivity, with FIG. 2 presenting the controllers as connecting via the network 200, and the subsequent hardware diagrams of FIG. 3A through 3C presenting these as a user interface connection, analogous to a keyboard, mouse, or joystick. It is understood that as long as the relevant communicative couplings are present somehow, whether it's WiFi, Bluetooth, radio signals, infrared, actual cables, or something else, as long as the systems are connected in a manner sufficient to practice the invention as described herein, the exact configuration is a logistical concern that one skilled in the art of systems administration or other disciplines of device connectivity might recognize as relatively trivial. It is further noted that the network 200 is portrayed entirely from a hardware perspective, with software aspects of network administration such as IP addressing, networking protocols, firewalls, network security, and so forth assumed and anticipated as present in accordance with best practices as known by one skilled in the art for providing a secure and functional network environment.

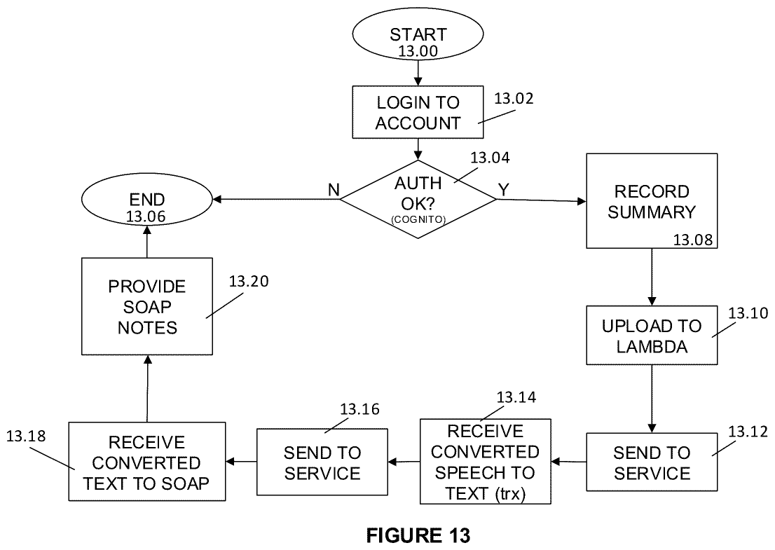

Referring now generally to the Figures, and particularly to FIG. 3A, FIG. 3A is a block diagram of the device 104 of the system 100 and displaying together both hardware and software aspects thereof, wherein the device 104 comprises: a central processing unit or “CPU” 104A; a user input module 104B; a display module 104C; a software bus 104D bi-directionally communicatively coupled with the CPU 104A, the user input module 104B, the display module 104C; the software bus 104D is further bi-directionally coupled with a network interface 104E, enabling communication with alternate computing devices by means of the network 100; and a memory 104F. The user input module 104B facilitates communication to external human-operated data sources such as the VR gear 108 and the optional force plate 106 if included, in addition to the basic utility of connection to a control element for the device 104 itself, such as connection to a keyboard and/or mouse. The software bus 104D facilitates communications between the above-mentioned components of the device 104. The network interface 104E may provide connection to a network such as the internet; network connectivity is neither included nor excluded as an aspect of the invention and the device 104 may be internet-enabled but need not be, except where this may concern connection to another system 100 device, such as a WiFi, Bluetooth, or similar wireless connection to the VR gear 108 or the optional force plate 106 if included in lieu of a cable. It is noted that wireless connections, particularly to the VR gear 108, may be preferable at least to facilitate use of the VR gear 108 without tangling the user 102 in cables, and it may also be preferred to conceal a force plate cable (not shown) or have a wireless connection to the optional force plate 106 also, to reduce potential trip hazards. It is further noted that a medical practitioner's office may have good reason to limit which in-office computers are internet-enabled at all, to make it easier to safeguard users' medical data and reduce the possibility of introducing any computer viruses to sensitive devices. The memory 104F of the device 104 includes a software operating system OP.SYS 104G. The software operating system OP.SYS 104G of the device 104 may be selected from freely available, open source and/or commercially available operating system software, to include but not limited to a.) a Z8 G4 computer workstation marketed by Hewlett Packard Enterprise of San Jose, California and running a Red Hat Linux™ operating system marketed by Red Hat, Inc. of Raleigh, North Carolina; (b.) a Dell Precision™ computer workstation marketed by Dell Corporation of Round Rock, Texas, and running a Windows™ 10 operating system marketed by Microsoft Corporation of Redmond, Wash.; (d.) a Mac Pro workstation running MacOS X™ as marketed by Apple, Inc. of Cupertino, Calif.; or (c.) other suitable computational system or electronic communications device known in the art capable of providing networking and operating system services as known in the art. The exemplary software program SW 104H consisting of executable instructions and associated data structures is optionally adapted to enable the device 104 to perform, execute and instantiate all elements, aspects and steps as required of the device 104 to practice the invented method in its various preferred embodiments in interaction with other devices of the network 100, including on many or all of the aspects the aspects of FIGS. 13 through 18.

The memory 104F may further include storage for data gathered in accordance with the invented method, a data storage 1041. The memory 104F may further include a machine learning algorithm 104J or model, such as for modeling the user 102 motion as indicated by the gathered data. The memory 104F may further include a speech-to-text utility software application 104K, such as for use in the processes of FIG. 13 through 17. The memory 104F may further include a text-to-SOAP utility software application 104L, such as for use in the processes of FIG. 13 through 17. The memory 104F may further include LAMBDA™ 104M, or other suitable software, software service, and/or web service known in the art such as for use in the processes of FIG. 13 through 17. The memory 104F may further include an authentication software 104N known in the art such as for use in the processes of FIG. 13 through 17, if a local software service is being utilized for this aspect; it is noted that, in several preferred embodiments, the Amazon Web Services Cognito™ service, as marketed by Amazon.com, Inc., of Seattle, WA, is preferred for this application, and this may be reflected by the authentication software 104N (whether local in the memory 104F or externally hosted) being referenced herein as “Cognito”.

Referring now generally to the Figures, and particularly to FIG. 3B, FIG. 3B is a block diagram of the VR gear 108 of the system 100 and displaying together both hardware and software aspects thereof, wherein the computing environment of the VR gear 108 (generally located within the VR headset 108A) includes: a central processing unit or “CPU” 108AA; a user input module 108AB; a display module 108AC; a software bus 108AD bi-directionally communicatively coupled with the CPU 108AA, the user input module 108AB, the display module 108AC; the software bus 108AD is further bi-directionally coupled with a network interface 108AE, enabling communication with other computing devices such as the device 104 of the system 100; and a memory 108AF. The user input module 108B facilitates communication to external human-operated data sources such as the left VR controller 108B and the right VR controller 108C, or other controller elements which may be used compatibly with the VR gear 108. The software bus 108AD facilitates communications between the above-mentioned components of the VR gear 108. The network interface 108AE may provide connection to a network such as the internet; network connectivity is neither included nor excluded as an aspect of the invention and the VR gear 108 may be internet-enabled but need not be, except where this may concern connection to another device, such as a WiFi, Bluetooth, or similar wireless connection to the device 104 or to the left VR controller 108B and/or the right VR controller 108C. It is noted that wireless connections, particularly to and from the VR gear 108, may be preferable at least to facilitate use of the VR gear 108 without tangling the user 102 in cables. The memory 108AF of the VR gear 108 may include a software operating system OP.SYS 108AG suitable for operating a virtual reality device as generally known in the art. It is noted that some current examples of virtual reality equipment currently available which could be suitable for use as the VR gear 108 might include the Oculus Quest 2 as marketed by Meta of Menlo Park, CA, United States; the Sony PlayStation VR as marketed by Sony of Tokyo, Japan; the Valve Index VR Kit as marketed by Valve of Bellevue, WA, United States; or the HP Reverb G2 as marketed by Hewlett Packard of Palo Alto, CA, United States. It is further generally noted that, though essentially specialized wearable computing devices, any virtual reality device may further include components, features, or elements not represented in the diagram of FIG. 3B, including proprietary elements particular to that brand or model of virtual reality console. It is further noted that the VR gear 108 need not be any of these commercially-available video-gaming consoles, and also that a similar device produced for a specific category of medical use instead of provided as a multifaceted consumer entertainment console could incorporate fewer frills and still retain the basic technological functionality necessary to practice of the invented method as described herein. The memory 102AF of the VR gear 108 may include at least one software program SW 108AH consisting of executable instructions and associated data structures optionally adapted to enable the VR gear 108 to perform, execute and instantiate all elements, aspects and steps as required of the VR gear 108 to practice the invented method in its various preferred embodiments in interaction with the rest of the system 100 as described. The memory 108AF may further include storage for data gathered in accordance with the invented method, a data storage 108AI. The memory 108AF may further include a machine learning algorithm 108AJ or model, such as for modeling the user 102 motion as indicated by the gathered data.

Referring now generally to the Figures, and particularly to FIG. 3C, FIG. 3C is a block diagram of the 2nd VR gear 202 of the network 200 of FIG. 2 and displaying together both hardware and software aspects thereof, wherein the computing environment of the 2nd VR gear 202 (generally located within a VR headset 202A) includes: a central processing unit or “CPU” 202AA; a user input module 202AB; a display module 202AC; a software bus 202AD bi-directionally communicatively coupled with the CPU 202AA, the user input module 202AB, the display module 202AC; the software bus 202AD is further bi-directionally coupled with a network interface 202AE, enabling communication with the network 200 or other devices; and a memory 202F. The user input module 202B facilitates communication to external human-operated data sources such as the left VR controller 202B and the right VR controller 202C, or other controller elements which may be used compatibly with the 2nd VR gear 202. The software bus 202D facilitates communications between the above-mentioned components of the 2nd VR gear 202. The network interface 202E may provide connection to a network such as the internet; network connectivity is neither included nor excluded as an aspect of the invention and the 2nd VR gear 202 may be internet-enabled but need not be, except where this may concern connection to another device, such as a WiFi, Bluetooth, or similar wireless connection to the network 200 or to the left VR controller 202B and/or the right VR controller 202C. It is noted that wireless connections, particularly to and from the 2nd VR gear 202, may be preferable at least to facilitate use of the 2nd VR gear 202 without tangling a user in cables. The memory 202F of the 2nd VR gear 202 may include a software operating system OP.SYS 202G suitable for operating a virtual reality device as generally known in the art. It is noted that some current examples of virtual reality equipment currently available which could be suitable for use as the 2nd VR gear 202 might include the Oculus Quest 2 as marketed by Meta of Menlo Park, CA, United States; the Sony PlayStation VR as marketed by Sony of Tokyo, Japan; the Valve Index VR Kit as marketed by Valve of Bellevue, WA, United States; or the HP Reverb G2 as marketed by Hewlett Packard of Palo Alto, CA, United States. It is further generally noted that, though essentially specialized wearable computing devices, any virtual reality device may further include components, features, or elements not represented in the diagram of FIG. 3C, including proprietary elements particular to that virtual reality console. It is further noted that the 2nd VR gear 202 need not be any of these commercially-available video-gaming consoles, and also that a similar device produced for a specific category of medical use instead of provided as a multifaceted consumer entertainment console could incorporate fewer frills and still retain the basic technological functionality necessary to practice of the invented method as described herein. The memory 102F of the 2nd VR gear 202 may include at least one software program SW 202H consisting of executable instructions and associated data structures optionally adapted to enable the 2nd VR gear 202 to perform, execute and instantiate all elements, aspects and steps as required of the 2nd VR gear 202 to practice the invented method in its various preferred embodiments in interaction with the rest of the network 200 as described. The memory 202F may further include storage for data gathered in accordance with the invented method, a data storage 2021. The memory 202F may further include a machine learning algorithm 202J or model, such as for modeling the user 102 motion as indicated by the gathered data.

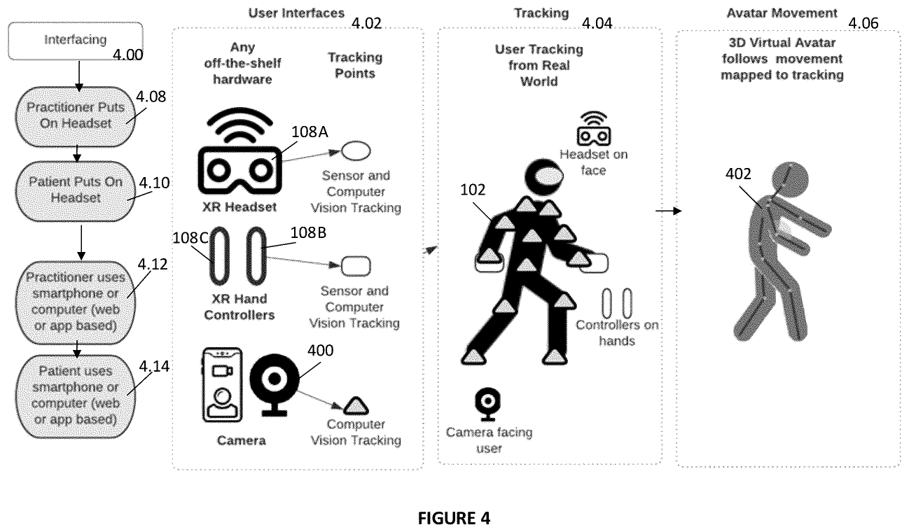

Referring now generally to the Figures, and particularly to FIG. 4, FIG. 4 is a first chart presenting various aspects of the invented method as practiced within the system 100, particularly pertaining to forming connections between user actions and digital modeling of the virtual reality gear. This chart can be organized into an Interfacing column 4.00 at the leftmost side of the chart, a User Interfaces column 4.02 second-to-left, a Tracking column 4.04 at the second-to-right, and an Avatar Movement column 4.06 at the rightmost side of the chart. Included under the Interfacing column 4.00: in sub-element 4.08, the practitioner or coach puts on a VR headset such as the 2nd VR headset 202A; in sub-element 4.10, the user 102 puts on a VR headset such as the VR headset 108A. Alternatively, some aspects of the invented method might be performable without full sets of VR equipment, and might use alternative equipment such as a computer, phone, or tablet. A smartphone in particular includes features for enabling augmented reality functions, such as gyroscopes and GPS. Therefore, also included under the Interfacing column 4.00: in sub-element 4.12, the practitioner or coach uses a smartphone or computer to implement a web or app based implementation, and in sub-element 4.14, the user 102 uses a smartphone or computer to implement a web or app based implementation. It is noted that depending upon the technological environment, these sub-elements might also be mix-and-matched, for instance if one party has VR gear but the other doesn't. In the User Interfaces column 4.02, a symbol key is provided for the visual presented in the Tracking column 4.04, specifically that a VR/AR/MR/XR headset, such as the VR headset 108A or the 2nd VR headset 202A, is represented as an oval; controllers such as the VR controllers 108B&C or the 2nd VR controllers 202B&C are represented as rounded rectangles; and body points detected by a camera 400 doing visual tracking are represented by triangles. It is noted that, while a camera was not presented in the hardware diagrams, a camera may be an additional input device utilized in the system 100, and may be accordingly communicatively coupled to an appropriate computing device as necessary for receiving and interpreting data generated by the camera 400 and utilizing the camera data in accordance with the method. In the Tracking column 4.04, an image of a user wearing a VR headset, holding controllers, and being tracked by a camera facing the user is presented, with the symbols as codified in the User Interfaces column 4.02 showing how the gear tracks the position of the user's body parts. In the Avatar Movement column 4.06, the body parts tracked in the Tracking column 4.04 are drawn in a virtual medium, such that the movement of the user's body as tracked is re-created in virtual rendering as a virtual avatar 402. It is noted that in this image the position of the virtual figure is not actually the same position as that of the user in the Tracking column 4.04, and the virtual avatar 402 is simply shown in some postural position; it is understood that the virtual avatar 402 may use the data as gathered according to this Figure to match the movement of a user as that user moves, or may alternatively be molded to present some other movement or position, such as to demonstrate to or guide the user 102 in performing a particular exercise or stretch. It is noted that the camera 400 is another optional hardware component that might not be utilized in all embodiments of the invented system, but may provide further possibilities for functionality and data gathering.

Referring now generally to the Figures, and particularly to FIG. 5, FIG. 5 is a second chart presenting various aspects of the invented method as practiced within the invented system of FIG. 2, particularly pertaining to the user 102 and coach interaction as processed through a virtual interface. In a first column 5.00 at the far left, the user 102 is interacting with the VR gear 108 as shown. In a second column 5.02, arrows show data input from the VR gear 108 being gathered. In a third column 5.04, Machine Learning Algorithms are applied to this received data, such as to analyze joint angles, joint torques, and center of mass. In a fourth column 5.06, further algorithms may be applied. In a fifth column 5.10, the virtual avatar 402 is instantiated, utilizing the data and analysis generated in the previous columns proceeding from the left of the Figure. In a rightmost column 5.12, a coach interacts with the 2nd VR gear 202 or another means of accessing the virtual avatar 402, allowing this coach to utilize an interface represented in a second-to-right column 5.14 to observe the movements of the user 102 as represented by the virtual avatar 402.

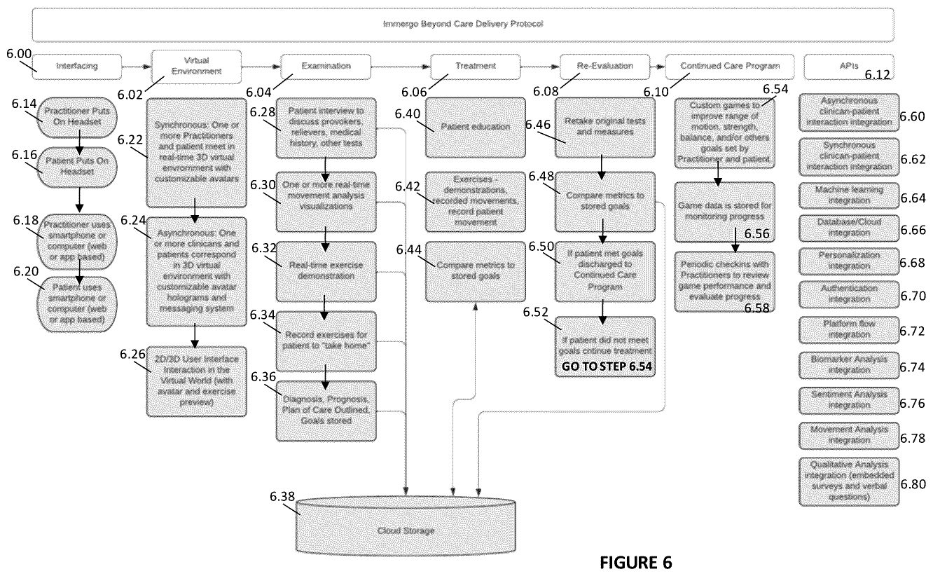

Referring now generally to the Figures, and particularly to FIG. 6, FIG. 6 is a third chart presenting various aspects of the invented method as practiced within the system 100. This chart presents an overview of an examination and treatment protocol incorporating the invented method for utilizing VR technology in coaching. As presented across the top, the elements of this chart can broadly be read as sub-elements of the following series of categories or steps: an Interfacing 6.00 step, a Virtual Environment 6.02 step, an Examination 6.04 step, a Treatment 6.06 step, a Re-Evaluation 6.08 step, a Continued Care Program 6.10 step, and an Application Programming Interfaces 6.12 (“APIs 6.12”) step. Included under Interfacing 6.00: in sub-element 6.14, the practitioner or coach puts on a VR headset such as the 2nd VR headset 202A; in sub-element 6.16, the user 102 puts on a VR headset such as the VR headset 108A. Alternatively, some aspects of the invented method might be performable without full sets of VR equipment, and might use alternative equipment such as a computer, phone, or tablet. A smartphone in particular includes features for enabling augmented reality functions, such as gyroscopes and GPS. Therefore, also included under Interfacing 6.00: in sub-element 6.18, the practitioner or coach uses a smartphone or computer to implement a web or app based implementation, and in sub-element 6.20, the user 102 uses a smartphone or computer to implement a web or app based implementation. It is noted that depending upon the technological environment, these sub-elements might also be mix-and-matched, for instance if one party has VR gear but the other doesn't. Under Virtual Environment 6.02, notable sub-elements include the following: in sub-element 6.22, a synchronous virtual meeting is conducted between one or more practitioners and the user 102, within a real-time customizable digital environment, while in sub-element 6.24, the same parties might asynchronously correspond; in sub-element 6.26, a user (such as a coach or the user 102) interacts individually with a user interface alone. Under Examination 6.04, some key sub-elements include the following. In sub-element 6.28, there is an interview to discuss provokers, relievers, medical history, and other tests may be discussed or performed. In sub-element 6.30, one or more real-time movement analysis visualizations may be performed. In sub-element 6.32, one or more real-time exercise demonstrations may be performed. In sub-element 6.34, one or more exercises for the user to ‘take home’ may be recorded. Sub-element 6.36 may include diagnosis, prognosis, plan of care outlining, and setting of goals. It is noted that any or all of these sub-elements of Examination 6.04 may be recorded in a cloud storage volume 6.38 for subsequent access, such as later reference. Under Treatment 6.06, some key sub-elements include the following. Sub-element 6.40 is education. Sub-element 6.42 is exercises, such as demonstrations, recorded movements, and recording of movement of the user 102. Sub-element 6.44 includes comparing metrics to stored goals, such as in reference to the goals set at sub-element 6.36 and stored in the cloud storage 6.38. Under Re-Evaluation 6.08, some key sub-elements may include the following. At sub-element 6.46, the user 102 may retake the original tests and measures. At sub-element 6.48, the metrics from the re-taking of tests might be measured against previously set and stored goals. At sub-element 6.50, if the user 102 has met the goals, the user 102 may be discharged to the Continued Care Program 6.10. In sub-element 6.52, if the user 102 did not meet the goals, treatment may continue. Under Continued Care Program 6.10, some key sub-elements may include the following. At sub-element 6.54, there may be custom games to improve range of motion, strength, balance, and/or other goals set by the user 102 and practitioner. At sub-element 6.56, game data is stored for monitoring progress. At step 6.58, there are periodic check-ins to review game performance and evaluate progress. Under APIs 6.12, some key sub-elements may include the following. At sub-element 6.60, there is asynchronous clinician-user interaction integration. At sub-element 6.62, there is synchronous clinician-user interaction integration. At sub-element 6.64, there is machine learning integration. At sub-element 6.66, there is database/cloud integration. At sub-element 6.68, there is personalization integration. At sub-element 6.70, there is authentication integration. At sub-element 6.72, there is platform flow integration. At sub-element 6.74, there is Biomarker Analysis integration. At sub-element 6.76, there is Sentiment Analysis integration. At sub-element 6.78, there is Movement Analysis integration. At sub-element 6.80, there is Qualitative Analysis integration (embedded surveys and verbal questions).

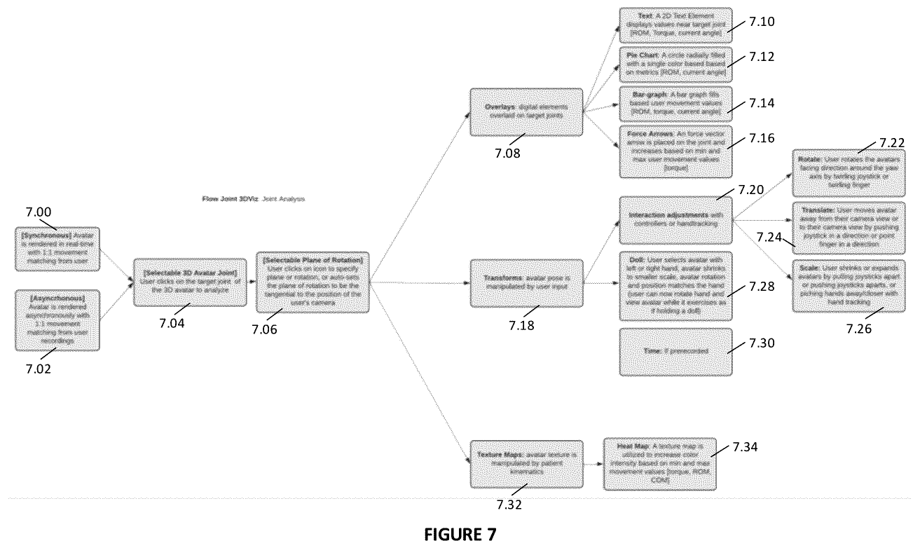

Referring now generally to the Figures, and particularly to FIG. 7, FIG. 7 is a fourth chart presenting various aspects of the invented method as practiced within the system 100, pertaining particularly to analysis and assessment of the joint health of the user 102. Elements 7.00 and 7.02 present two alternative methods for the movement of the user 102 to be observed utilizing the VR gear 108. In element 7.00, the movement of the user 102 is rendered in real-time, as the user 102 moves, and the virtual avatar 402 generated by the VR gear 108 imitates the detected motion of the user 102 in a 1:1 correspondence, such that the virtual avatar 402 moves the same way the user 102 moves, specifically enough for irregularities in the movement of the user 102 to be observed by a coach working with the user 102. Alternatively, the movement of the user 102 may be demonstrated asynchronously, such that the movement of the user 102 is recorded by the VR gear 108 and can be played back later, as re-enacted by the virtual avatar 402, for observation. It is noted that the distinction between these two options is only whether the movement demonstration is being done ‘live’ or being recorded, and further that, while there are unique benefits to real-time interaction, a movement demonstration done ‘live’ could also be recorded for review later, such as for a coach to study further offline, for the coach and user 102 to consider together from a third-person vantage-point (i.e. “see how your knee moved right there?”), or for a comparison later on to show improvement in the movement of a user 102. In element 7.04, regardless of how the motion data was obtained, the virtual avatar 402 generated to imitate the motions of the user 102 may be queryable, such that the user 102 or the coach can click or tap on parts of the virtual avatar 402 to view more information or analysis. For instance, if the knee of the virtual avatar 402 makes a certain motion of interest, a user could click on the virtual knee to learn more: view the raw data that led to the knee being rendered that way (very useful for debug), find out which device or combination of devices (such as the VR headset 108A, the left VR controller 108B, the right VR controller 108C, the optional force plate 106, etc.) detected the motion, or even view an analysis of why the computer ‘thinks’ this motion occurred and what it might mean about other structural elements' motion. For instance, most people might have experienced the common problem that if one has an injury (or even just muscle tension) in one leg, the other side may work harder to compensate; if one injures their left foot or leg, that affects their right leg also, not to mention their hips and back, which may do a lot of shifting of weight or posture to take stress off the unsteady left leg; all this can happen without the user 102 even noticing, and soon the ‘left-leg’ injury has somehow expanded into full-body achy soreness as well. That dynamic of shifting posture to take work away from the injured left leg would show up in the movement of the user 102, and in this example, the sore right hip (for instance) of the user 102 might be queryable: ‘Why is the right hip moving that way? Because the left hip is moving this way. Why is the left hip doing that? Because the left leg can't support the weight. Oh, well we know why that is, the left ankle got sprained.’ Indeed, it can be very difficult, particularly with longstanding chronic pain, to figure out what is actually an injury (if it isn't obvious), and what other complaints (such as that sore right hip) might have arisen as an indirect result, caused by the body of the user 102 compensating for, working around, or protecting the injured body part. In element 7.06, a user viewing the motion of the virtual avatar 402, such as a coach or the user 102, can control view perspective, such as presenting the motion from a specified rotational angle or zooming in or out. It is noted that providing flexibility of view perspective for observation and manipulation of a 3D virtual environment is well known at least in the art of computer graphics and video gaming, and one skilled in the art will appreciate both what view options may be appropriate or preferred, and the importance of providing these. At element 7.08, various overlays may be provided for further viewing options, such as digital elements overlaid on target joints. It is noted that digital environments such as video games also routinely include this feature, where an overlay view may be toggled to visualize a particular element more easily. It is noted that the overlay view may specifically provide information about a selected element, such as a data readout when a user clicks on and queries a virtual body part as described above, but that an overlay may also be a more generalized map for visualizing the whole system, such as relevant color-coding, a mapping of pictures of internal muscle structure or bone structure as understood by medicine onto a current postural position, or an ability for a coach to “mark up” or annotate the image. It is understood that those overlay features specifically mentioned here should not be construed as limiting, but rather as accessible examples. In element 7.10, some text is included as part of the overlay view, which may display values such as the ROM (range of motion), torque, or current angle. In element 7.12, a pie chart is included as an overlay element, namely a circle radially filled with a certain color based on a metric such as ROM or current angle. In element 7.14, a bar graph may be included as an overlay element for displaying values such as the ROM, torque, or current angle. In element 7.16, one or more force arrows may be overlaid on the virtual avatar 402 image to highlight aspects of the motion of the user 102. It is further noted that these overlays might be applied in real-time, such that the user 102 can even watch the overlays interpret or annotate their moves as they make them, or might be applied to recorded motion being played back asynchronously. Alternatively or additionally, element 7.18 includes transformations, namely the pose of the avatar being directly manipulated by user input other than the motion input generated by the user 102, such as input by a coach to show a movement to the user 102. Element 7.20 is a further elaboration on the Transformations of element 7.18, specifically Interaction Adjustments utilizing controllers or hand tracking, such as a coach manipulating a pose of the virtual avatar 402 with their VR controller or with a similarly tracked motion of their hand. Further elaborating on that, element 7.22 indicates that a user such as the user 102 or a coach might rotate the avatar's facing direction around the yaw axis by twirling a joystick, a finger, or some other control. It is noted that the term ‘yaw’ is used here as a term of art regarding three-dimensional rotation, and this term is most commonly used in the context of aerospace; as an accessible example, a plane in flight might rotate itself in these three dimensions: rolling (i.e. a ‘barrel roll’), pitching (i.e. pointing its nose upward or downward), or yawing (i.e. pointing its nose to one side or the other to change direction of travel). Element 7.24 indicates that the location of the virtual avatar 402 might also be adjusted, either by ‘drag and dropping’ the virtual avatar 402 elsewhere in the virtual setting, or by adjusting the camera, and suggests that a joystick or finger control might be used to implement this. It is noted that no limitation should be construed regarding which particular controls or key bindings may be assigned for performing which actions, except as stated in the claims. It is further noted that controls may vary depending on what interface is available to a user, just as someone playing a video game utilizing a joystick will utilize a different set of controls than one using a keyboard and mouse to play the same game might use. Element 7.26 represents a third option, namely scaling the size of the virtual avatar 402. Element 7.28 presents an additional or alternative option for transforming the virtual avatar 402, namely an option to turn the avatar into a ‘doll’ version that can be freely molded to show demonstrated motions, as though a user were holding a doll. Element 7.30 notes that time may also be a relevant dimension in this manipulation, particularly if the motion is prerecorded; for instance, the recording might be slowed down for a ‘slow-motion replay’. It is noted that, since the discipline of kinesiology and movement coaching over all is concerned particularly with how the body of the user 102 moves, and with adjusting that movement where necessary to improve the health of the user 102 such as by building habits of moving differently, it may be a common tool in coaching practice for a coach to demonstrate a posture or stretch for the user 102 to imitate, or to observe the movement of the user 102 and manually adjust how the user 102 is positioned. This physical component can make telehealth physical therapy a particular challenge to the coach and the user 102, and giving these tools ‘back’ to physical coaching as practiced remotely, by providing a virtual equivalent as described above, might be considered a key benefit of the invention. Element 7.32 further mentions the feature of texture maps, i.e. mapping overlays onto the virtual avatar 402 which reflect aspects of the movement of the user 102. Element 7.34 further elaborates that this might be for instance a heat map, where color intensity or hue is mapped onto the virtual avatar 402 based on gathered movement data, such as based on minimum and maximum values of torque, ROM, or COM. This visualization might make it easier for the user 102 and/or a coach to see patterns and points of interest regarding the movement of the user 102.

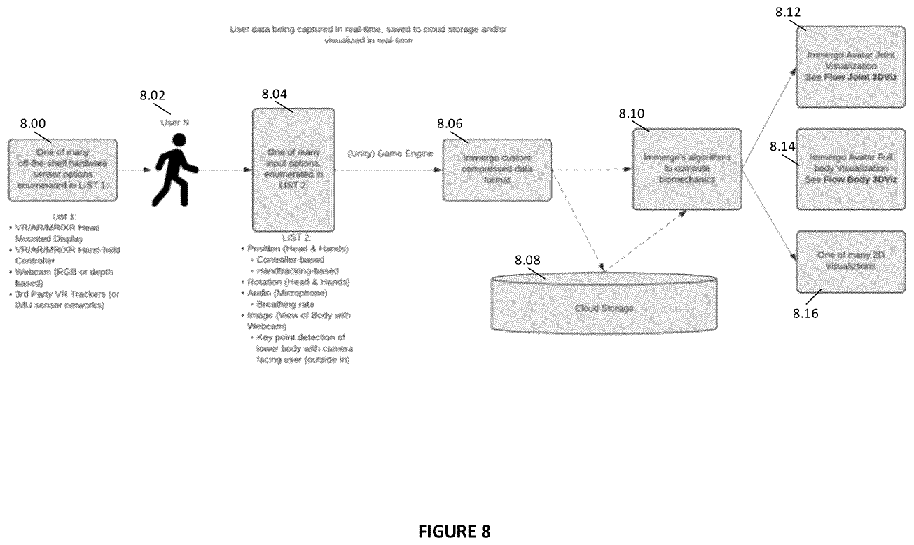

Referring now generally to the Figures, and particularly to FIG. 8, FIG. 8 is a fifth chart presenting various aspects of the invented method as practiced within the system 100, pertaining particularly to capturing user data over time. In element 8.00, a suitable hardware environment is established, such as by utilizing items from the following list: VR/AR/MR/XR Head Mounted Display, VR/AR/MR/XR Hand-held Controller, Webcam (RGB or depth based), 3rd Party VR Trackers (IMU sensor networks). It is noted that this aspect is represented in the earlier hardware diagrams as the VR gear 108, and that the VR headset 108A and VR controllers 108B&C, and that any sort of device considered suitable as known in the art might be utilized, of which all these are just examples. In element 8.02, a user N, such as the user 102 or a coach, puts on, starts up, or otherwise interfaces with the equipment of element 8.00. In element 8.04, the user N provides input to the equipment of element 8.00, such as by moving parts of their body that the equipment is coupled to or held by, moving parts of their body otherwise visible to the equipment (such as moving one's hand within view of a motion-tracking camera interface), speaking or breathing within range of audio equipment such as a microphone, and so on as presented in LIST 2 adjacent to element 8.04. The input generated by the user N in element 8.04 may be introduced within the software structure of a game engine (such as a game engine constructed in Unity). At element 8.06, the received input is encoded in a proprietary data compression format. At element 8.08, the received and encoded input might be stored in Cloud Storage. At element 8.10, regardless of whether the data was also stored in Cloud Storage, algorithms are applied to compute biomechanics. At element 8.12, a visualization of a detected joint such as an elbow or knee may be generated based on the previously processed input data. At element 8.14, a full body visualization may be generated. At element 8.16, one of many 2D visualizations might be generated.

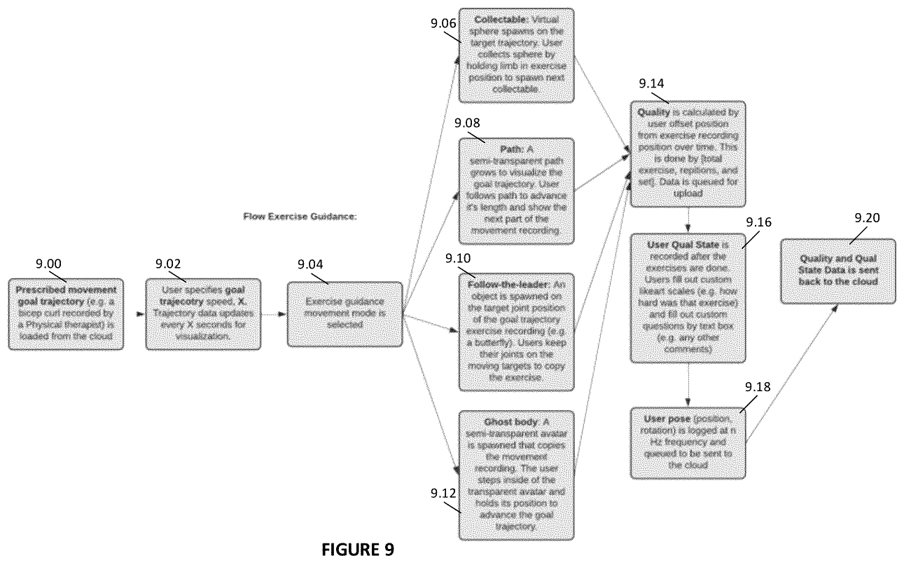

Referring now generally to the Figures, and particularly to FIG. 9, FIG. 9 is a sixth chart presenting various aspects of the invented method as practiced within the system 100, particularly pertaining to flow exercise guidance. At element 9.00, a prescribed movement goal trajectory is loaded, such as from cloud storage; this may be an item of recorded movement data, such as a bicep curl demonstration recorded by a coach. At element 9.02, a goal trajectory speed X is specified by a user, which sets an update of the visualization with current trajectory data every X seconds. At element 9.04, an exercise guidance movement mode is selected. At element 9.06, a ‘collectable’ exercise format may be instantiated, such that a user such as the user 102 is tasked to retrieve a virtual object such as a generated virtual sphere; for instance, if the user 102 needs practice in bending down, the sphere may appear near the floor, requiring the user 102 to practice their bending down to retrieve the sphere. At element 9.08, a ‘path’ exercise format may be instantiated, such that a user such as the user 102 is presented with a semi-transparent path visualization, and tasked to trace the presented path with their input motion. The path may be shown only partially at a time, and extended as the exercise progresses. At element 9.10, a ‘follow-the-leader’ exercise format may be instantiated, such that an object appears in the virtual environment that a user such as the user 102 is tasked to follow the movements of. At element 9.12, a ‘ghost-body’ exercise may be instantiated, wherein a semi-transparent avatar appears in the virtual environment that copies the movement recording, such as a demonstrated movement provided by a coach, and a user such as the user 102 is tasked to step inside the semi-transparent avatar and move such that their detected body remains inside the semi-transparent avatar body. At element 9.14, following an exercise, the quality of the exercise performance by the user 102 is assessed. At element 9.16, the quality assessment is recorded, and the user 102 may be queried about how hard they found the exercise to complete, or asked to provide other comments. At element 9.18, the current pose or position of the user is logged at n Hz frequency, and logged to be sent to the cloud or other data storage. At element 9.20, the recorded data is sent to the cloud or other data storage.

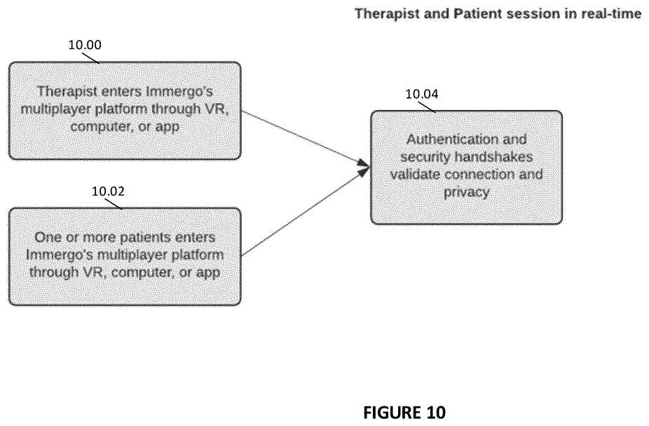

Referring now generally to the Figures, and particularly to FIG. 10, FIG. 10 is a seventh chart presenting various aspects of the invented method as practiced within the system 100, particularly pertaining to a coach and user session. In element 10.00, a coach enters a virtual multiplayer platform through virtual reality, augmented reality, a computer, an application, or other suitable means known in the art. In element 10.02, the user 102, or even multiple users, enter a virtual multiplayer platform through virtual reality, augmented reality, a computer, an application, or other suitable means known in the art. In element 10.04, authentication and security handshakes validate connection and privacy for all of these connections.

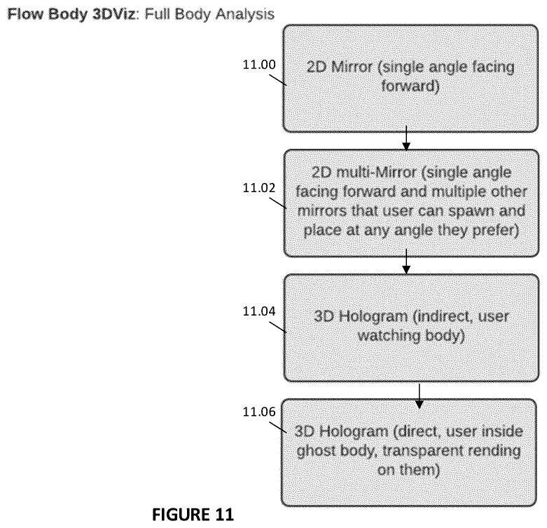

Referring now generally to the Figures, and particularly to FIG. 11, FIG. 11 is an eighth chart presenting various aspects of the invented method as practiced within the system 100, particularly pertaining to full body analysis. Each element represents an alternative possible view option for presentation of the virtual avatar 402. In element 11.00, a two-dimensional mirror view is presented, which shows the subject from one angle facing forward, as though the subject were looking into a full-length wall mirror. In element 11.02, a two-dimensional multi-mirror option is presented, which may include or consist of a single angle facing forward view and multiple other ‘mirrors’ that can be generated and placed at other angles to show other views, like the multiple mirrors one might see and utilize in a fitting room for instance. In element 11.04, the option is presented of a three-dimensional hologram view, which may comprise or include a reflection of the subject's whole body from a third-person perspective, as though the subject were watching someone else. In element 11.06, a ‘ghost body’ option is presented, wherein a three-dimensional avatar body is projected around the viewpoint of the user, as though the user were wearing a virtual suit of armor. It is noted that these are just some non-limiting examples of interest, and that other view options and useful ways to render the virtual avatar 402 are possible as well.

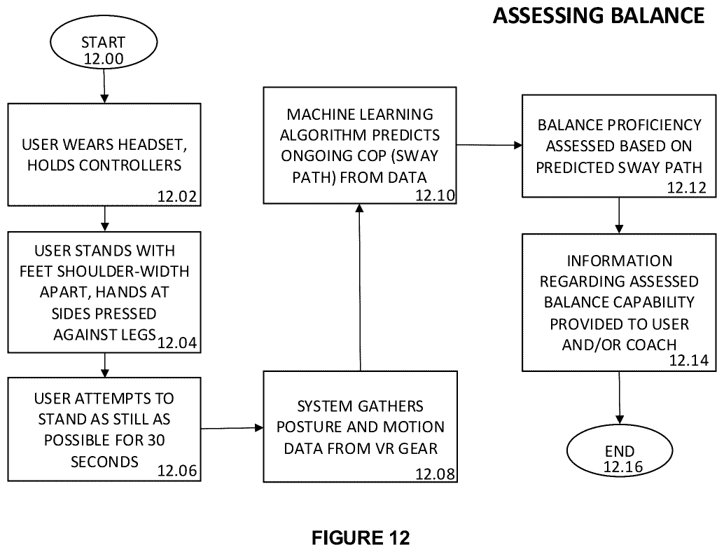

Referring now generally to the Figures, and particularly to FIG. 12, FIG. 12 is a process chart presenting one preferred procedure for assessing a user's balance capability utilizing the system 100. At step 12.00, the process starts. At optional step 12.02, the user 102 wears the VR headset 108A, holds the left VR controller 108B with their left hand, and holds the right VR controller 108C with their right hand. At step 12.04, the user 102 stands with their feet shoulder-width apart and their hands at their sides pressed against the sides of their legs. At step 12.06, the user 102 attempts to stand as still as they are able to. At step 12.08, while the user 102 stands still, the system 100 measures any motion of the body of the user 102, such as shifting off balance, swaying, and so on; even if the posture of the user 102 may appear solid to a human observer the VR gear 108 may pick up minute weight shifts or patterns or compensations in balance that could indicate information about the posture and balance of the user 102, and even indicate how likely the user 102 may be to lose their balance in less ideal circumstances. It may even be possible to determine patterns from this gathered data, such as whether the user 102 might favor one side or another, which structural elements of the body of the user 102 may be receiving too much stress (such as a weaker muscle on one side than the other, which could result in a certain pattern of faltering or compensation), or similar, when interpreted by one skilled in the art of kinesiology, such as a coach, who knows what to look for. At step 12.10, the machine learning algorithm 104J predicts the ongoing center of pressure based on the gathered data. At step 12.12, the user's balance proficiency is assessed based on the gathered data, particularly how much the user 102 might be expected to sway and how, as predicted by the machine learning algorithm 104J from how much and how the user 102 sways in the gathered data. In step 12.14, this information is provided to the user 102 and coach, to be acted upon. At step 12.16, the process ends.

Referring now generally to the Figures, and particularly to FIG. 13, FIG. 13 is a first process chart presenting a first invented process for synthesizing recorded data from a coaching session into notes for use by a coach. At step 13.00, the process starts. At step 13.02, a coach logs into an account accessible with the device 104 or via the network 100. At step 13.04, the login is authenticated by using a suitable authenticating web service, such as Cognito, if access is not authorized by the coach's interaction with the authenticating software, software service or web service, then the process of FIG. 13 is ended unsuccessfully at step 13.06. Otherwise, at step 13.08, data is recorded from a coaching session conducted by the coach, such as but not limited to written notes, audio data, force plate data, video data, and VR software program data. At step 13.10, the collected data is uploaded to Lambda for secure storage. It is noted that therapeutic coaching contexts such as physical therapy may include data protocols specific to the field of medicine, such as careful management of PHI in compliance with HIPAA. At step 13.12, one or more selected data elements are sent to a speech-to-text service, and at step 13.14, one or more text elements (such as files in the format *.trx) as converted from speech elements by the speech-to-text service is received in response. At step 13.16, one or more data elements including text converted from speech, is sent to a service for converting text to SOAP format, and at step 13.18, notes in SOAP format are received as a product of this conversion. At step 13.20, the SOAP notes are provided to the coach. At step 13.06, the process ends.

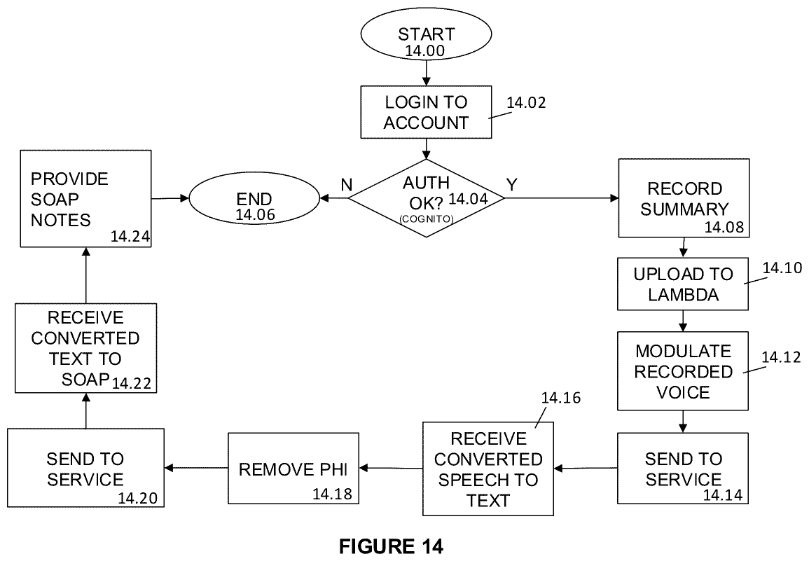

Referring now generally to the Figures, and particularly to FIG. 14, FIG. 14 is a second process chart presenting a second invented process for synthesizing recorded data from a training session into notes for use by a coach. At step 14.00, the process starts. At step 14.02, an authorized individual, such as a coach, logs into an account accessible with the device 104 or via the network 100. At step 14.04, the login is authenticated using a suitable authentication software or web service known in the art; if access is not authorized by the coach's interaction with the authenticating software, software service or web service, then the process of FIG. 14 is ended unsuccessfully at step 14.06. Otherwise, at step 14.08, data is recorded from a training session conducted by the coach, such as but not limited to written notes, audio data, force plate data, video data, and VR software program data. At step 14.10, the collected data may be uploaded to Lambda for secure storage. At step 14.12, audio data to be converted speech-to-text is first modulated, such as to anonymize the voices in the audio data for additional security. At step 14.14, one or more selected data elements are sent to a speech-to-text service, and at step 14.16, one or more text elements (such as files in the format *.trx) as converted from speech elements by the speech-to-text service is received in response. At step 14.18, PHI is removed. At step 14.20, one or more data elements including text converted from speech, is sent to a service for converting text to SOAP format, and at step 14.22, notes in SOAP format are received as a product of this conversion. At step 14.24, the SOAP notes are provided to the coach. At step 14.06, the process ends.

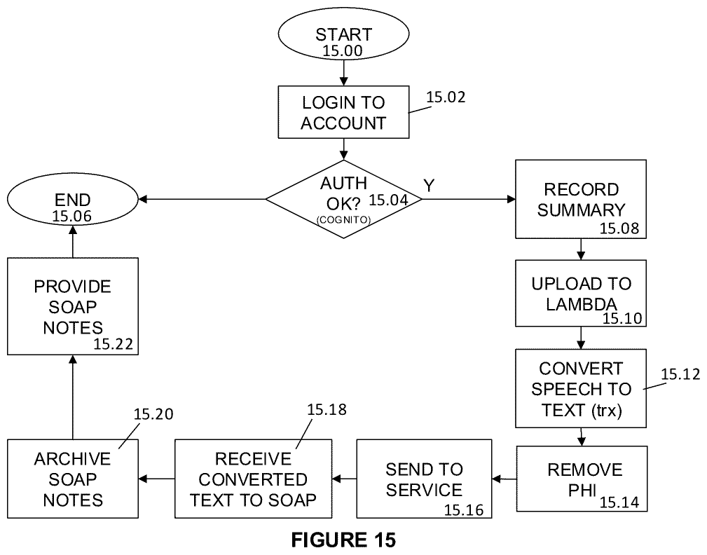

Referring now generally to the Figures, and particularly to FIG. 15, FIG. 15 is a third process chart presenting a third invented process for synthesizing recorded data from a training session into notes for use by a coach. At step 15.00, the process starts. At step 15.02, a coach logs into an account accessible with the device 104 or via the network 100. At step 15.04, the login is authenticated using a suitable authenticating software, software service, and/or web service, again such as Cognito; if access is not authorized by the coach's interaction with the authenticating software, software service or web service, then the process of FIG. 15 is ended unsuccessfully at step 15.06. Otherwise, at step 15.08, data is recorded from a training session conducted by the coach, such as but not limited to written notes, audio data, force plate data, video data, and VR software program data. At step 15.10, the collected data is uploaded to Lambda for secure storage. At step 15.12, audio data is converted by speech-to-text algorithm; it is noted that one of the distinctions between this chart and the charts of FIGS. 13 and 14 is that the speech-to-text conversion is completed without use of a third-party service. At step 15.14, PHI is removed. At step 15.16, one or more data elements including text converted from speech, is sent to a service for converting text to SOAP format, and at step 15.18, notes in SOAP format are received as a product of this conversion. At step 15.20, the SOAP notes may be optionally archived; it is noted that the chart of FIG. 15 mentions this optional step specifically, but other processes of FIGS. 13 through 17 might also incorporate this optional step as preferred. At step 15.22, the SOAP notes are provided to the coach. At step 15.06, the process ends.

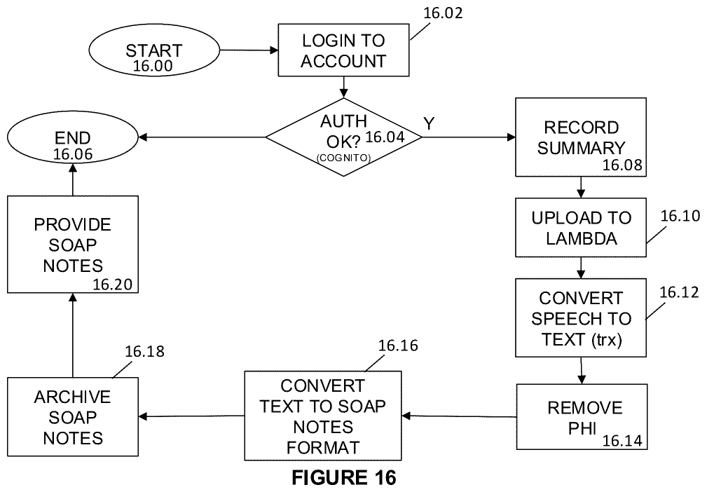

Referring now generally to the Figures, and particularly to FIG. 16, FIG. 16 is a fourth process chart presenting a fourth invented process for synthesizing recorded data from a training session into notes for use by a coach. At step 16.00, the process starts. At step 16.02, a coach, logs into an account accessible with the device 104 or via the network 100. At step 16.04, the login is authenticated using a suitable authenticating software, software service, and/or web service, again such as Cognito; if access is not authorized by the coach's interaction with the authenticating software, software service or web service, then the process of FIG. 16 is ended unsuccessfully at step 16.06. Otherwise, at step 16.08, data is recorded from a training session conducted by the coach, such as but not limited to written notes, audio data, force plate data, video data, and VR software program data. At step 16.10, the collected data is uploaded to Lambda for secure storage. At step 16.12, audio data is converted by speech-to-text algorithm; it is noted that one of the distinctions between this chart and the charts of FIGS. 13 and 14 is that the speech-to-text conversion is completed without use of a third-party service. At step 16.14, PHI is removed. At step 16.16, one or more data elements including text converted from speech, is converted from text to SOAP format; it is noted that one of the distinctions between this chart and the charts of FIGS. 13, 14, and 15 is that the text-to-SOAP conversion is completed without use of a third-party service. At step 16.18, the SOAP notes may be optionally archived; it is noted that the chart of FIG. 16 mentions this optional step specifically, but other processes of FIGS. 13 through 17 might also incorporate this optional step as preferred. At step 16.20, the SOAP notes are provided to the coach. At step 16.06, the process ends.

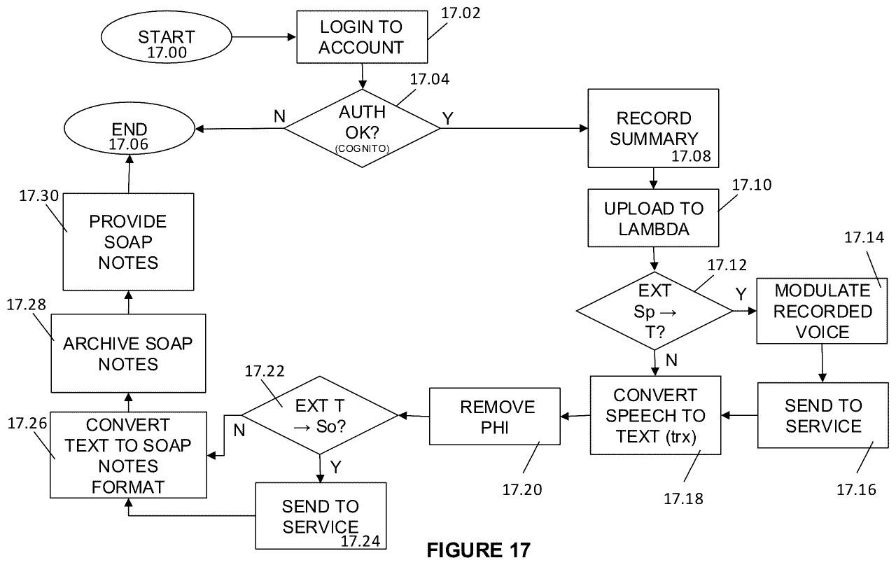

Referring now generally to the Figures, and particularly to FIG. 17, FIG. 17 is a fifth process chart presenting a fifth invented process for synthesizing recorded data from a training session into notes for use by a coach. At step 17.00, the process starts. At step 17.02, a coach logs into an account accessible with the device 104 or via the network 100. At step 17.04, the login is authenticated using a suitable authenticating software, software service, and/or web service, again such as using a suitable authenticating software, software service, and/or web service, again such as Cognito; if access is not authorized by the coach's interaction with the authenticating software, software service or web service, then the process of FIG. 17 is ended unsuccessfully at step 17.06. Otherwise, at step 17.08, data is recorded from a training session conducted, performed or experienced by the coach, such as but not limited to written notes, audio data, force plate data, video data, and VR software program data. At step 17.10, the collected data is uploaded to Lambda for secure storage. At step 17.12, it is decided whether an external text-to-speech converter is being utilized; if so, then at step 17.14 a voice modulator is used to distort audio data prior to sending the audio to the service at step 17.16. At step 17.18, the speech is converted to text and returned, either locally or by an external service. At step 17.20, PHI is removed. At step 17.22, it is determined whether an external text-to-SOAP service is being used. If so, the text data is sent to the service at step 17.24; either way, text is converted to SOAP at step 17.26. At step 17.28, the SOAP notes may be optionally archived; it is noted that the chart of FIG. 17 mentions this optional step specifically, but other processes of FIGS. 13 through 17 might also incorporate this optional step as preferred. At step 17.30, the SOAP notes are provided to the coach. At step 17.06, the process ends.

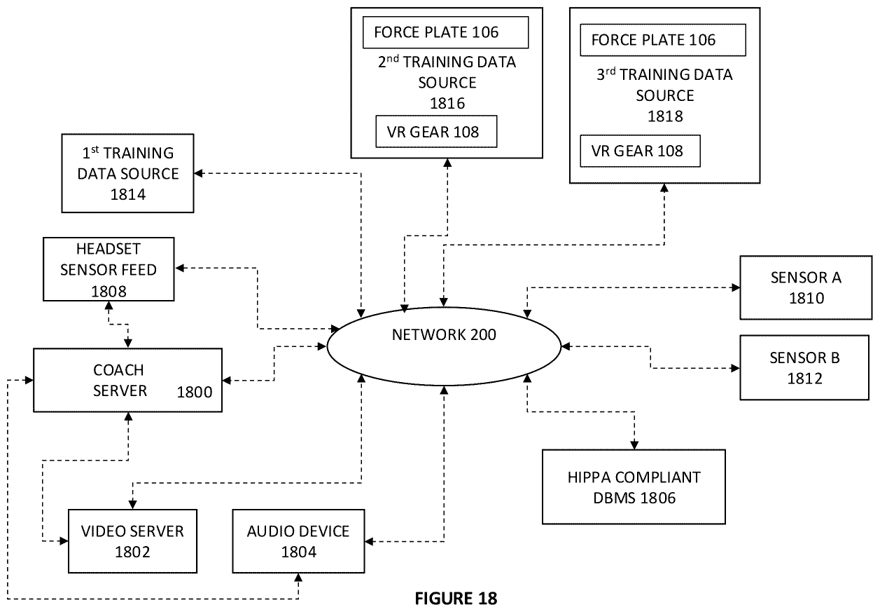

Referring now generally to the Figures, and particularly to FIG. 18, FIG. 18 is a network diagram of an enhanced embodiment of the information technology network 200 and by which the one or more, or all of the aspects of the processes for synthesizing recorded data of FIG. 13 through FIG. 17 are practiced. The network 200 may further include a coach server 1800; a video server 1802; an audio device 1804; a database management system 1806; a headset sensor feed 1808; a first sensor 1810; and a second sensor 1812. The first sensor 1810 and the second sensor 1812 might be any kind of sensor, such as but not limited to an electrocardiogram; an electroencephalogram; a camera; a spirometer; an Upright GO 5 system as marketed by Upright Technologies LTD. of Tel Aviv, Israel; a dynamometer; a BioDex system as marketed by Biodex Medical Systems, Inc. of Shirley, NY; wearable tech such as a FitBit as marketed by Google of Mountain View, CA, an Apple Watch™ wireless-networked digital watch as marketed by Apple, Inc. of Cupertino, CA, or similar. It is noted that the database management system 1806 might preferably be HIPAA-compliant, particularly if utilized in a medical context such as physical therapy. Further included on the network 1800 are a first training data source 1814, a second training data source, and a third training data source 1818, representing one or more network locations of laboratories or similar which comprise at least one each training source providing a VR gear 108 and optionally one force plate 106 that gather and originate training data, such as by following the method of FIG. 21.

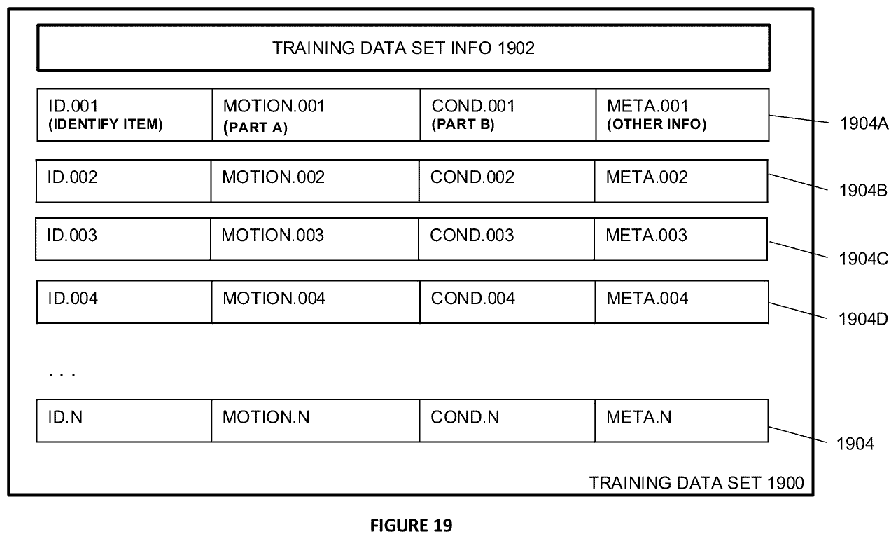

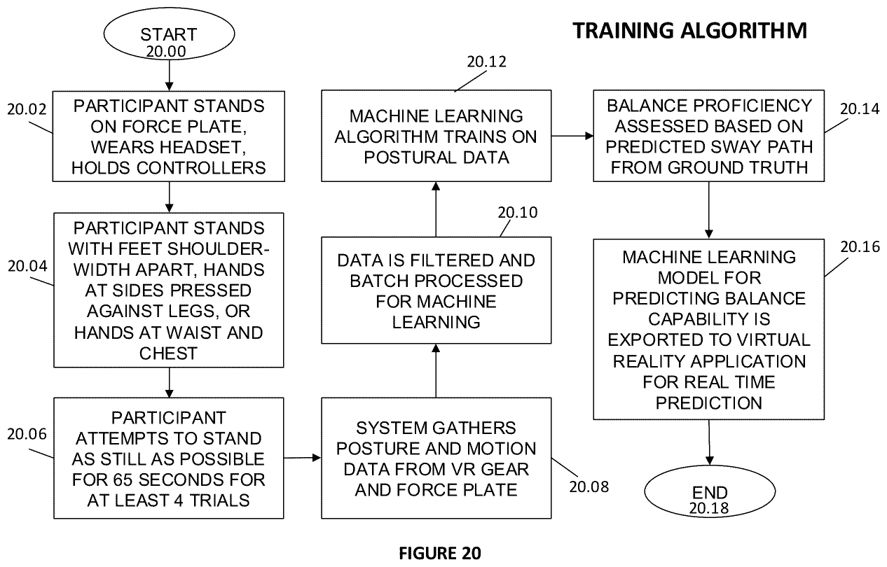

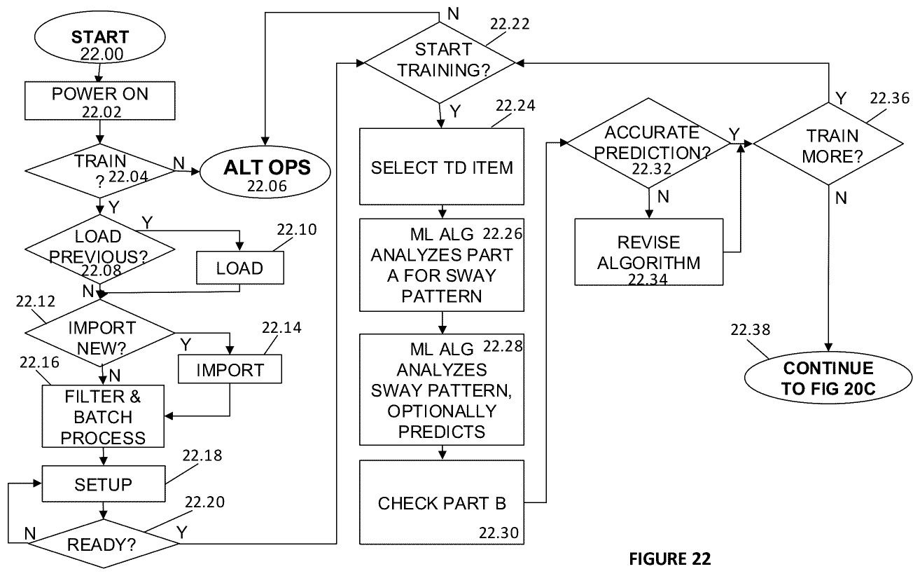

Referring now generally to the Figures, and particularly to FIG. 19, FIG. 19 is a block diagram representing a training data set 1900 (“the training data 1900”) for use in the machine learning algorithm training process of FIGS. 20 through 23. The training data 1900 might be stored in memory such as at the data storage 1041, and used to train the machine learning algorithm 104J. The training data set 1902 may be labeled or otherwise include additional identifying information regarding the training data 1902, particularly if multiple instances of the training data set 1900 are stored (such as different groups of training data 1900 for different purposes). It is noted that the training data 1900 is a representation of one representative set or group of a plurality of training data items 1902, and it is possible to have multiple groupings or subgroupings of training data 1900. The training data 1900 comprises a plurality of training data items 1904, represented herein examples of a first training data item 1904A numbered 001, a second training data item 1904B numbered 002, a third training data item 1904C numbered 003, and a fourth training data item 1904D numbered 004; an additional generic training data set item numbered N is also represented to show that this listing of items might be any length. Each training data item 1904 may include an identifier data field ID.N such as for storing a unique identifier (such as but not limited to an I.D. number or similar) for cataloging and accessing the associated item. Each training data item 1904 may further include a motion data field MOTION.N, such as for storing raw data regarding bodily motion as gathered and measured in FIG. 20. Each training data item 1904 may further include a condition data field COND.N for containing an assessed bodily condition associated with the data of the motion data field MOTION.N. It is noted that the motion data field MOTION.N is the “part A” of a training data pair as discussed in FIG. 22, and the condition data field COND.N the associated “part B” or training data label. Each training data item 1904 may further include a data field for other information, a metadata field META.N. Some examples of useful metadata to attach to individual training data items 1904 as preferred might include tracking when this item was generated, by what lab or data source, whether this item has been used yet in a given training cycle, a text label for identifying the item if this is preferred alongside an ID number, and so on. It is noted that, while the name of the person who was measured to produce the data might naturally be considered as another possible metadata item, to the extent that the gathered motion data is medical data, this may be contraindicated and data anonymity preferable.

Referring now generally to the Figures, and particularly to FIG. 20, FIG. 20 is a process chart presenting a preferred method for training a machine learning model. At step 20.00, the process starts. At optional step 20.02, the user 102 stands on the optional force plate 106 (if included), wears the VR headset 108A, holds the left VR controller 108B with their left hand, and holds the right VR controller 108C with their right hand. It is noted that step 20.02 may be optional as a result of some or all of the stated equipment being not in use or unavailable, such as an application wherein VR gear is used but a force plate is not, vice versa, or another system for detecting and recording motion is utilized, such as a camera. It is further noted that it is preferred that the training data 1900 such as for training the machine learning algorithm 104J be generated using the additional input of the optional force plate 106, as this may be higher-quality, more nuanced data. At step 20.04, the user 102 stands with their feet shoulder-width apart and their hands at their sides pressed against the sides of their legs, or with their hands at their waist and chest. At step 20.06, the user 102 attempts to stand as still as they are able to, such as but not limited to, for at least four trials having durations within the range of from 5 seconds to 3 minutes, or other suitable time duration as known in the art. At step 20.08, while the user 102 stands still, the system 100 measures motion of the body of the user 102, such as shifting off balance, swaying, and so on, and gathers data on the posture and motion of the user 102. The motion measurement of step 20.08 may comprise detections and separate or combined measurements of headset movement/rotation, controller movement/rotation, and instantiation of the center of mass body data of the user 103 indicating where the detected weight of the user 102 is centralized over the force plate 106.

Even if the posture of the user 102 may appear solid to a human observer, the optional force plate 106 (if included), VR gear 108, or other motion capture devices may pick up minute weight shifts or patterns or compensations in balance that could convey minute but important information about the posture and balance of the user 102, and even indicate how likely the user 102 may be to lose their balance in less ideal circumstances. It may even be possible to determine patterns from this gathered data, such as whether the user 102 might favor one side or another, which structural elements of the body of the user 102 may be receiving too much stress (such as a weaker muscle on one side than the other, which could result in a certain pattern of pain, faltering, compensation, or similar stress indicators), when interpreted by a well-trained machine learning model or one skilled in the art of kinesiology, such as a coach, who knows what to look for. At step 20.10, the gathered data is filtered and batch-processed for machine learning. At step 20.12, the machine learning algorithm 104J trains on the gathered data, such that the machine learning algorithm 104J is proficient in predicting a sway path (i.e., pattern of slight motion while trying to stand still) of the body of the user 102. At step 20.14, the balance proficiency of the user 102 is preferably assessed on the basis of comparison of the sway path relative to a ground truth orientation (i.e., the center of mass data of the user 102 dynamically measured by the force plate 106) with a sway path predicted by the machine learning algorithm 104J. It is understood that the term “ground truth” here further indicates the gold standard of center of mass measurement as known in the art, i.e., a measurement of a center of mass of the user 102 by a suitable clinical grade force plate 106 known in the art and commonly used in the art to assess balance.

At step 20.16, the machine learning algorithm 104J as trained for prediction of balance capability is exported to a virtual reality application for providing sway path predictions in real time. It is noted that one potential application of this process is for one or more different instances of the machine learning algorithm 104J to be trained by observing the sway paths of individuals already known to have certain physical conditions or ailments, or to be healthy, such that the machine learning algorithm 104J learns to recognize and distinguish both “healthy” sway path patterns and sway path patterns characteristic of certain ailments, and this analytic proficiency can then be used as a resource to assess new users whose condition is unknown. For instance, the machine learning algorithm 104J might learn to identify a first sway pattern characteristic of a physical injury such as a strained leg muscle or ankle sprain influencing a user's posture and balance, as distinct from a second sway pattern characteristic of a user's balance being impaired by a proprioceptive issue such as intoxication or a concussion, and both of these as distinct from a third, “normal” sway pattern generated by a user whose balance and posture is not modified by injury or impairment.

Referring now generally to the Figures, and particularly to FIG. 21, FIG. 21 is a first process chart presenting further detail regarding the training data 1900 generation steps 20.00 through 20.08. At step 21.00, the process starts. At step 21.02, all devices of the system 100 are powered on. At step 21.04, the VR gear 108 is initialized; besides powering on, this step might include any setup such as starting a relevant software process, performing any software updates, and generally getting the VR gear 108 ready to use as discussed in this process. At step 21.06, the optional force plate 106 is initialized; since this is a process for generating the training data 1900, it is noted that use of the optional force plate 106 is preferred and advised in this context. At step 21.08, the device 104 is initialized for recording and storing data generated by this process. At step 21.10, any other setup steps necessary to get the system 100 ready to perform the process of FIG. 21 are performed; this is intended as a “miscellaneous catch-all” step and may include routine maintenance on any of the devices of the system 100 which may happen to be due, fixing any problems which may arise, performing software updates, and anything else one skilled in the art of using computers generally knows to do when setting up a computing device to perform a task or process. As of step 21.12, the system 100 should be ready to perform the process of steps 21.14 through 21.46; if so, the process continues, and if not, setup continues until the system 100 is fully set up. At step 21.14, the user 102 whose motion is being measured to produce the training data 1900 is assessed by an expert such as a coach or medical professional; as the training data 1900 which is being generated includes categorization of the condition of the user 102 associated with the data (e.g. no balance issues, sprained left ankle, hip injury, Alzheimer's affecting balance, and so on) so that the machine learning algorithm 102J can be trained to associate a detected sway pattern with a probable condition, the training data 1900 may preferably comprise paired sets of (A.) motion data and (B.) condition. At step 21.16, the user 102 stands on the optional force plate 106. At step 21.18, the user 102 wears the VR headset 108A and grips the VR controllers 108B&C. At step 21.21, the user 102 is instructed about how the assessment will proceed, and instructed to stand in the posture of step 21.22. It is noted that, in a laboratory setting being used for data gathering, a coach or technician might brief the user 102 verbally as to what to expect and how the user 102 should stand for the assessment, but written instructions or an instructional video played on a display of the system 100 (such as the VR headset 108A the user 102 may already be wearing) might also be used. In step 21.22, the user 102 stands with feet shoulder-width apart, hands at sides pressed against legs, or hands at waist and chest, as instructed. At step 21.24, the user 102 is ready to begin the assessment. At step 21.26, it is decided whether to start; if not, return to step 21.24 to ensure the user 102 is ready to proceed. If so, the process continues to step 21.28, wherein the user 102 attempts to stand as still as possible for the duration of the assessment. It is noted that living beings generally are never perfectly still; breathing, slight shifts in posture and balance, and other small physical motions all are constantly ongoing, and can reveal a lot about the physical condition of the user 102, such as but not limited to whether the user 102 stands “favoring” one side of the body (which could indicate pain or an injury there), or sways slightly in place, or has difficulty completing the task of standing unsupported without stumbling or tilting to the side. At step 21.30, while the user 102 stands still, the system 100 measures and records the subtle movements of the body of the user 102 as discussed; for instance, the VR headset 108A may detect movement of the head of the user 102, the VR controllers 108B&C may detect motion of the hands or arms, and the optional force plate 106 may detect shifts in balance and weight placement. At step 21.32, it is verified that the data has been recorded and is sound. If not, then at step 21.34, it is determined whether to fix the error and retry; it is noted that this might be a computer bug to fix, a failure of the user 102 to follow directions such that the data is invalidated, or something else. If it is decided not to fix the problem and retry, the process may end at step 21.36 with continuing to alternative operations. Otherwise, the issue is fixed at step 21.38, and the trial is retried starting from step 21.24. If the data was sound, then a trial has been completed; the same user 102 may be measured over multiple “rounds” for a more accurate read, such as but not limited to a sequence of four 65-second trials. At step 21.40, it is decided whether to repeat, such as for additional trials. Once all trials for this user 102 have been completed, at step 21.42, the training data produced from this session is stored appropriately, such as in the data storage 1041 or another suitable location to be accessed by the process of FIG. 22. At step 21.44, it is determined whether to “loop” and repeat the process with further additional users, or to conclude this session of generating training data. Once the training data has been generated, the process proceeds to step 21.46 to continue to FIG. 22 and train the machine learning algorithm 102J with the training data that has been generated. It is noted that, particularly in a collaborative operation, the continuity between FIGS. 21 and 22 may not be linear; one could continue gathering more training data even while using yesterday's gathered training data to ongoingly train the machine learning algorithm 102J.

Referring now generally to the Figures, and particularly to FIG. 22, FIG. 22 is a second process chart continuing from FIG. 21, presenting further detail regarding the machine learning algorithm training steps 20.10 through 20.14. At step 22.00, the process starts. At step 22.02, the device 104 is powered on. Any other devices useful or necessary for performing the method of FIG. 23 as discussed herein may also be powered on and otherwise made or kept available, such as a server stack providing storage or processing power, network utilities providing access to other devices, or devices on the network which may have recently generated new training data items 1904, such as by following the process of FIG. 21, such that these new training data items 1904 can be obtained by the device 104 and used in the present process. At step 22.04, it is determined whether a machine learning training process is initiated. If not, the device 104 may proceed to alternate ops at step 22.06, such as other unrelated processes. If a machine learning training process is starting, then at step 22.08, it is decided whether to load previous data, such as training data 1900 which has already been used to train the machine learning algorithm 104J previously, or old version(s) of the machine learning algorithm 104J to build on or modify. If so, whatever old data is preferred is loaded at step 22.10. At step 22.12, it is determined whether to import new data, such as by accessing training data 1900 which was freshly generated by the process of FIG. 21; it is noted that this may entail accessing other devices, such as a server providing data storage or whichever laboratory devices gathered the training data 1900. At step 22.14, if so, the new data is imported. At step 22.16, the training data 1900 (as now gathered together by the previous loading and importing into a single set of training data 1900 to be used for the current machine learning training session) is filtered and batch-processed. At step 22.18, any other required setup may be performed; as of step 22.20, the device 104 is ready to perform a machine learning training session. At step 22.22, it is determined whether to start training. If not, the device 104 may proceed to alternative operations. If so, then at step 22.24, one of the training data items 1904 is selected for a cycle of the training process; as a representative non-limiting example, this description selects the first training data item 1904A. At step 22.26, the machine learning algorithm 104J analyzes the recorded motion data of MOTION.001, and determines a sway pattern. In later stages of training the machine learning algorithm 104J, the machine learning algorithm 104J may also make a prediction as to COND.001—i.e. a prediction about the condition of the user 102 whose data recording session (per FIG. 20) generated the first training data item 1904A—and be assessed for accuracy. At step 22.30, the actual COND.001 is revealed, i.e. the “right answer” to the user 102 condition assessment, as generated by an expert in step 21.14. It is noted that in early stages of machine learning training, it's often considered best practice to start by providing a set of example data for the machine learning algorithm 104J to start out with, such that the machine learning algorithm 104J has a “knowledge base” to find patterns in and “draw conclusions” from about subsequent new data. In step 22.32, if the machine learning algorithm 104J is being assessed for accuracy and made a prediction that was incorrect, i.e. produced the “wrong answer”, then at step 22.34, the machine learning algorithm 104J may be revised. It is noted that, in accordance with general practices for training and generating machine learning algorithms, this revision may consist of modifying criteria, instructing the algorithm differently, incorporating more data for the machine learning algorithm 104J to use for reference material, or selecting among multiple versions of the machine learning algorithm 104J for those versions which produce the highest accuracy of prediction. It is noted that a key aspect of machine learning technology is that the machine learning algorithm 104J is a “black box” function—ideally, the machine learning algorithm 104J takes in data and predicts correct answers to an acceptable level of accuracy, and how the machine learning algorithm 104J “decides” which answer is likely to be correct, which characteristics of the data are relevant signifiers, is beside the point as long as the machine learning algorithm 104J answers accurately enough. Accordingly, one generally starts out by training the machine learning algorithm 104J to correlate a pattern of data (i.e. a part A) with a condition (i.e. a part B), and what characteristics of part A's reliably correlate to different part B's (i.e. “a sprained ankle wobbles like this, but a concussion wobbles like that”); then moves on to assessing the training proficiency of the machine learning algorithm 104J by how accurately the machine learning algorithm 104J synthesizes an accurate part B given a part A to analyze. Finally, the machine learning algorithm 104J in actual use would generate a prediction concerning an unknown part B based on a received novel part A. At step 22.36, it is determined whether the machine learning algorithm 104J should be trained further. It is noted that training is only ever complete relative to a designated standard of accuracy, and may reach a point of increasingly diminishing returns; once the machine learning algorithm 104J accuracy is high enough, the machine learning algorithm 104J may be ready for implementation “in the real world”, such that the process continues to FIG. 23 in step 22.38.

Referring now generally to the Figures, and particularly to FIG. 23, FIG. 23 is a third process chart continuing from FIG. 22, presenting further detail regarding application of a trained machine learning algorithm to assess an unknown user as discussed in FIG. 20. At step 23.00, the process starts. At step 23.02, the VR gear 108 of the user 102 is powered on. Any other devices useful or necessary for performing the method of FIG. 23 as discussed herein may also be powered on and otherwise made or kept available, such as any remote servers from which the VR gear 108 may receive software updates, particularly including software updates to the software 108AH or the machine learning model 108AJ, or any routers or other utilities which allow the VR gear 108 to receive such updates. At step 23.04, it is decided whether the VR gear 108 should perform a software update; if so, at step 23.06, a software update is performed. At step 23.08, any other setup steps necessary to get the system 100 ready to perform the process of FIG. 23 are performed; this is intended as a “miscellaneous catch-all” step and may include any other setup the VR gear 108 may require for use, such as but not limited to starting up a relevant software process or adjusting the headset strap fit. As of step 23.10, the VR gear 108 should be ready to use; if so, the process continues, and if not, setup continues until the system 100 is fully set up. It is noted that use of a more sophisticated setup of the system 100, such as one including the optional force plate 106, is preferred for data gathering implementations such as generating the training data 1900 of FIG. 21, but a more minimal setup, such as that of FIG. 1B, may be preferred for individual home use or private use by a coach and trainee, to download or remotely access software services and already-trained machine learning models. Whatever version of the system 100 is being used, that system is fully set up as of step 23.10. At step 23.12, it is determined whether an assessment of the user 102 is the current activity; if not, the VR gear 108 may proceed to alternate ops at step 23.14, such as other VR gear 108 programs or applications which could be related to the invented method but may also not be. At step 23.16, the user 102 wears the VR headset 108A and grips the VR controllers 108B&C. At step 23.18, the user 102 is instructed about how the assessment will proceed, and instructed to stand in the posture of step 23.20. It is noted that, since the method of FIG. 23 may be practiced “at home”, outside of a laboratory setting, with just the user 102 and the VR gear 108 belonging to the user 102, it would be useful for the software performing this assessment to include a manual or a visual display with instructions for the user 102 to follow to complete the assessment correctly, but a coach could also instruct the user 102 what to do in the context of a training session or similar. In step 23.20, the user 102 stands with feet shoulder-width apart, hands at sides pressed against legs, or hands at waist and chest, as instructed. At step 23.22, the user 102 is ready to begin the assessment. At step 23.24, it is decided whether to start; if not, return to step 23.16 and repeat any necessary steps, or do anything else which may be needed, to ensure the user 102 is ready to proceed. If so, the process continues to step 21.26, wherein the user 102 attempts to stand as still as possible for the duration of the assessment. At step 21.28, while the user 102 stands still, the system 100 measures and records the subtle movements of the body of the user 102 as discussed. At step 21.30, it is verified that the data has been recorded and is sound. If not, then at step 21.32, it is determined whether to fix the error and retry; it is noted that this might be a computer bug to fix, a failure of the user 102 to follow directions such that the assessment is invalidated, or something else. If it is decided not to fix the problem and retry, the process may end at step 21.34 with continuing to alternative operations. Otherwise, the issue is fixed at step 21.36, and the trial is retried starting from step 21.24. If the data was sound, then a trial has been completed; the user 102 may be measured over multiple “rounds” for a more accurate read, such as but not limited to a sequence of four 65-second trials. At step 21.38, it is decided whether to repeat, such as for additional trials. Once all trials have been completed, at step 21.40, the data produced from this session is stored appropriately, such as in the data storage 108AI or another suitable location. At step 23.42, the VR gear 108 or a remote server such as the device 104 may conduct additional analysis of the data which was just generated, such as analysis utilizing the machine learning algorithm 104J which was trained in FIG. 22. It is noted that in certain embodiments the machine learning algorithm 104J may be downloaded to the VR gear 108, and this processing may be done either locally or remotely, depending. If machine learning analysis is not elected, the process may end at step 23.48. Otherwise, the machine learning algorithm 104J receives the data generated in steps 23.28, detects a sway pattern at step 23.44, and, with whatever degree of certainty the trained AI is capable of, at step 23.46, predicts a likely condition of the user 102 based on the detected sway pattern. It is noted that being analyzed by an automated software program is no substitute for diagnosis and care by a medical professional, and the AI is unlikely to always be right even when trained exhaustively on a wide variety of data, but informal detection of possible ailments may still be useful and valuable even so, such as providing data-supported guidance for a conversation between the user 102 and a coach or doctor.

While selected embodiments have been chosen to illustrate the invention, it will be apparent to those skilled in the art from this disclosure that various changes and modifications can be made herein without departing from the scope of the invention as defined in the appended claims. For example, the size, shape, location or orientation of the various components can be changed as needed and/or desired. Components that are shown directly connected or contacting each other can have intermediate structures disposed between them. The functions of one element can be performed by two, and vice versa. The structures and functions of one embodiment can be adopted in another embodiment, it is not necessary for all advantages to be present in a particular embodiment at the same time. Every feature which is unique from the prior art, alone or in combination with other features, also should be considered a separate description of further inventions by the applicant, including the structural and/or functional concepts embodied by such feature(s). Thus, the foregoing descriptions of the embodiments according to the present invention are provided for illustration only, and not for the purpose of limiting the invention as defined by the appended claims and their equivalents.

We claim:

1. A method for training and applying a machine learning algorithm to assess and predict physical health of a human user based on measurement of physical motion of the human user, the method comprising:
assessing and categorizing a physical health state of a sample user;
providing a first motion capture system comprising a force plate, a virtual reality headset, and two handheld virtual reality controllers;
instructing the sample user to stand on the force plate, wear the virtual reality headset, and grip the two handheld virtual reality controllers;
instructing the sample user to stand as still as the sample user is able to, for a plurality of short durations of time, while the motion capture system records a set of sample user motion data regarding slight bodily motion of the sample user even while the sample user is attempting to stand still;
filtering and batch processing the set of sample user motion data;
training a machine learning algorithm to predict a sway pattern of the sample user based on the set of sample user motion data;
training a machine learning algorithm to associate the sway pattern of the set of sample user motion data as characteristic for the physical health state of the sample user as previously assessed;
exporting the machine learning algorithm as trained to a virtual reality application; and
applying the machine learning algorithm as trained to predict an unknown physical health state of a new user based on assessment of a new user sway pattern in a received motion data set associated with the new user.

2. The method of claim 1, wherein the assessment of the new user is done remotely via an electronic communications network.

3. The method of claim 2, wherein the output of the virtual reality headset, and two handheld virtual reality controllers is received when in use by the new user and analyzed by application of the machine learning algorithm.

4. The method of claim 1, wherein the sample user is a physical wellness client.

5. The method of claim 1, wherein the new user is a kinesthetic performer.

6. The method of claim 1, wherein the new user is acting under advice of a coach.

7. The method of claim 1, further comprising:
assessing and categorizing a physical health state of each of a plurality of sample users;
providing a same motion capture system serially to each of the plurality of sample users, the motion capture system comprising a force plate, a virtual reality headset, and two handheld virtual reality controllers;
instructing each sample user to separately and individually stand on the force plate, while wearing one the motion capture system, and separately grip the two handheld virtual reality controllers;
instructing each sample user to stand as still as each sample user is able to, for a plurality of short durations of time, while the motion capture system records comprising a set of sample user motion data regarding slight bodily motion of each sample user even while each sample user is attempting to stand still and monitored by the motion capture system; and
filtering and batch processing the set of sample user motion data.

8. The method of claim 1, further comprising adding at least one set of sample data that is generated by use of at least one sample user of an alternate motion capture system, the alternate motion capture system comprising an alternate force plate, an alternate virtual reality headset, and two alternate handheld virtual reality controllers.

9. The method of claim 1, further comprising:
assessing and categorizing a physical health state of each of a plurality of sample users;
providing a plurality of motion capture systems comprising a force plate, a virtual reality headset, and two handheld virtual reality controllers to each of the sample users;
instructing each sample user to separately and individually stand one of the plurality of force plates, while wearing one of the plurality of motion capture systems, and grip the two handheld virtual reality controllers of said one of the plurality of motion capture systems;
instructing each sample user to stand as still as each sample user is able to, for a plurality of short durations of time, while one of said plurality of motion capture system records one of a set of sample user motion data regarding slight bodily motion of each sample user even while each sample user is attempting to stand still; and
filtering and batch processing the set of sample user motion data.

10. The method of claim 1, further comprising the virtual reality headset rendering a same virtual reality session to at least two sample users while sample data is recorded.

11. The method of claim 10, wherein the virtual reality session is derived from the virtual reality application.

12. The method of claim 10, further comprising the virtual reality headset rendering the same virtual reality session to the new user while data is recorded.

13. The method of claim 12, wherein the virtual reality session is derived from the virtual reality application.

14. A client device comprising:
an augmented reality user set (“the user set”), the user set comprising a headset and an additional positional feedback device, the positional feedback device comprising a plurality of body element positional sensors (“the plurality of sensors”) communicatively coupled with the headset, wherein the user set is configured to be worn by a human user and to generate and transmit relative body part dynamic positional information describing a dynamic kinesiologic action of the user's body;
one or more processors bi-directionally communicatively coupled by a communications module with the user set; and
a memory bi-directionally communicatively coupled by the communications module with the one or more processors and the user set, the memory storing software executable instructions executing on the client device, the software executable instructions when executed by the one or more processors cause the client device to:
a. access a video segment and direct the client system to dynamically visually render a user avatar derived from and dynamically responsive to kinesiologic relative body element positional information generated by and received from the plurality of sensors;
b. transmit to the headset a sequence of data frames from a data stream program, the data stream program, the data stream program presenting at least one personalized body parts movement pathway (“the pathway”) indicating at least one recommended kinesiologic path of at least two anatomical elements of the user, wherein the sequence of data frames provides kinesiologic and positioning information of a modeling avatar for rendering by the headset, the modeling avatar adapted to dynamically present to the user via the headset aspects of the at least one personalized movement pathway; and
c. display an interactive dynamic overlay of the modeling avatar over the user avatar by the headset, the interactive overlay displayed in association with a plurality of dynamically updated kinesiologic body part positional information received by the headset from the plurality of sensors, wherein the dynamically updated kinesiologic body part positional information generated by the user set is derived from the plurality of dynamically kinesiologic body part positional information generated by the plurality of sensors of the positional feedback device and received and integrated into the user avatar by the one or more processors.

15. The method of claim 14, wherein the video segment comprises a sequence of athletic movement images.

16. The method of claim 14, wherein the video segment comprises a sequence of human performance movement images.

17. The method of claim 16, wherein the sequence of human performance movement images express a choreographed pattern of human movement.

18. The method of claim 14, wherein the client device is accessed by a human user for a health evaluation.

19. The method of claim 14, wherein the client device is accessed by a human user for a movement training session.

20. The method of claim 14, wherein the client device is accessed by a human user for a performance training session.
